(19) United States
(12) Patent Application Publication
(10) Pub. No.: US 2025/0018180 A1
(43) Pub. Date: Jan. 16, 2025

(54) DEVICES AND TECHNIQUES FOR INTERFERENTIAL CURRENT STIMULATION

(71) Applicant: The Joan and Irwin Jacobs Technion-Cornell Institute, New York, NY (US)

(72) Inventors: Charles RODENKIRCH, New York, NY (US); Michael JIGO, New York, NY (US); Qi WANG, New York, NY (US); Jason CARMEL, New York, NY (US)

(73) Assignee: THE JOAN AND IRWIN JACOBS TECHNION-CORNELL INSTITUTE, NEW YORK, NY (US)

(21) Appl. No.: 18/770,995

(22) Filed: Jul. 12, 2024

Related U.S. Application Data
(60) Provisional application No. 63/513,256, filed on Jul. 12, 2023.

(51) Int. Cl. A61N 1/32; A61N 1/04; A61N 1/36

(52) U.S. Cl. CPC A61N 1/323; A61N 1/0456; A61N 1/0492; A61N 1/36034

(57) ABSTRACT

Devices and techniques for inferential current stimulation. A device includes a power source, multiple pairs of electrodes, and a controller. Each pair of electrodes is adapted to apply current from the power source in a current path to stimulate a subcutaneous target. The current paths overlap at an interference point which is a result of a summation of the electrical fields of the current delivered via the pairs of electrodes. The controller is configured to send control signals in order to control the current delivered from the power source to each pair of electrodes. The controller is also configured to control the current delivered to each pair of electrodes such that frequencies of the current paths are higher than a frequency of the summation of the electrical fields and the waveform resulting from the summation of the electrical fields includes discrete interference pulses delivered with periods of quiescence between pulses

### CROSS-REFERENCE TO RELATED APPLICATIONS

This application claims the benefit of U.S. Provisional Application No. 63/513,256 filed on Jul. 12, 2023, the contents of which are hereby incorporated by reference.

### TECHNICAL FIELD

The present disclosure relates generally to enhancing perception of sensory content, and more specifically to enhancing perception of sensory content via vagus nerve stimulation.

### BACKGROUND

Impaired sensory processing is linked to a variety of negative effects on life. Examples include withdrawal from social situations, increased risk of falls or other accidents, and inability to live independently. Seniors often face these challenges and more as sensory processing declines with age. Aging both deteriorates sensory receptors such as eyes and ears, and impairs the brain's ability to process signals received from those receptors (i.e., central sensory processing). The combined effects of sensory loss may cause anxiety, depression, and accelerated onset of dementia.

In addition to aging, other individuals may experience impaired sensory processing. Individuals with sensory processing disorders such as attention deficit hyper disorder (ADHD) and dyslexia have been shown to be more likely to have impaired sensory processing. Moreover, fatigue, inattention, and concussions can all impair sensory processing. Athletes and other active individuals can therefore also experience impaired sensory processing. Decreased sensory acuity is linked with increased risk of injury and poor performance at work, school, and recreational activities.

Existing solutions for treating sensory loss in seniors such as glasses and hearing aids address deterioration of receptors. However, other these solutions do not address other potential causes of impaired sensory processing. Additionally, these solutions face challenges in improving sensory processing for individuals who do not demonstrate deteriorated receptors. Other existing solutions may include chemical or other pharmaceutical stimulants which can help improve sensory processing but have side effects such as cardiac damage, insomnia, anxiety, loss of appetite, and addiction. Further, nootropics and supplements may fail to provide the benefits advertised at best, and be actively harmful to consumers at worst.

Neural circuits in the body can be readily activated by being exposed to certain electrical fields. Further, electrical energy delivered to other targets in the body can also have beneficial effects (e.g., organs, muscles, etc.). It has been shown that differences in the shape of the electrical field waveforms interacting with the tissue can result in positive or negative consequences on ability to reliably, safely, and chronically activate neurons or create beneficial effect. Therefore, it is desirable to be able to deliver accurate electrical field waveforms to targets located subcutaneously.

In the human body, many stimulation targets (e.g., peripheral nerves like the vagus nerve) are located superficially enough under the skin that transcutaneous electrical stimulation can reach the nerve. However, the complexities of electrical current propagation through heterogeneous organic tissues makes accurately targeting of precise waveforms transcutaneously to neural targets difficult, especially with increasing depth of target.

Further, for many neural targets, the transcutaneous electrical current must first pass through superficial layers of tissue (e.g., skin, muscle, etc.) for which activation is undesired as activated off target nerves in these superficial layers can create uncomfortable sensations or distracting muscle contraction.

New solutions for delivering accurate and precise electrical waveforms transcutaneously to subcutaneous neural targets and solutions for minimizing activation of off-target regions would therefore be desirable.

### SUMMARY

A summary of several example embodiments of the disclosure follows. This summary is provided for the convenience of the reader to provide a basic understanding of such embodiments and does not wholly define the breadth of the disclosure. This summary is not an extensive overview of all contemplated embodiments, and is intended to neither identify key or critical elements of all embodiments nor to delineate the scope of any or all aspects. Its sole purpose is to present some concepts of one or more embodiments in a simplified form as a prelude to the more detailed description that is presented later. For convenience, the term “some embodiments” or “certain embodiments” may be used herein to refer to a single embodiment or multiple embodiments of the disclosure.

Certain embodiments disclosed herein include a device. The device comprises: a power source; a plurality of pairs of electrodes having a plurality of current paths, wherein each pair of electrodes is electrically connected to the power source, wherein each pair of electrodes is adapted to apply a current delivered from the power source in a respective current path of the plurality of current paths in order to stimulate a subcutaneous target within a subject, wherein the plurality of current paths have a plurality of first waveforms with a plurality of first frequencies, wherein the plurality of current paths overlap at an interference point having a second waveform with a second frequency, wherein the second waveform is result of a summation of electrical fields of the current delivered via each pair of electrodes; and a controller, wherein the controller is communicatively connected to the power source, wherein the controller is configured to send control signals in order to control the current delivered from the power source to each pair of electrodes in order to create a stimulation pattern for each electrode pair, wherein the controller is further configured to control the current delivered to each pair of electrodes such that each of the plurality of first frequencies is higher than the second frequency and such that the second waveform includes discrete interference pulses delivered with periods of quiescence between the interference pulses.

Certain embodiments disclosed herein include any of the devices, computer readable mediums, or systems, noted above or below, wherein the current delivered via each pair of electrodes is realized via electrical signals that are between 200 and 2000 microseconds in length.

Certain embodiments disclosed herein include any of the devices, computer readable mediums, or systems, noted above or below, wherein the second waveform is charge balanced.

Certain embodiments disclosed herein include any of the devices, computer readable mediums, or systems, noted above or below, wherein an interference pattern generated via the overlap of the plurality of current paths of the plurality of pairs of electrodes is a biphasic waveform.

Certain embodiments disclosed herein include any of the devices, computer readable mediums, or systems, noted above or below, wherein the biphasic waveform includes an interphase gap.

Certain embodiments disclosed herein include any of the devices, computer readable mediums, or systems, noted above or below, wherein the second waveform has a total pulse width of 200 to 2000 microseconds.

Certain embodiments disclosed herein include any of the devices, computer readable mediums, or systems, noted above or below, wherein the total pulse width of the second waveform is 1 millisecond.

Certain embodiments disclosed herein include any of the devices, computer readable mediums, or systems, noted above or below, wherein the discrete stimulation pulses of the interference pattern is delivered at a frequency between 15 and 60 Hertz.

Certain embodiments disclosed herein include any of the devices, computer readable mediums, or systems, noted above or below, wherein the discrete stimulation pulses of the interference pattern is delivered at a frequency below 100 Hertz.

Certain embodiments disclosed herein include any of the devices, computer readable mediums, or systems, noted above or below, wherein each of the plurality of first frequencies is above a predetermined threshold.

Certain embodiments disclosed herein include any of the devices, computer readable mediums, or systems, noted above or below, wherein the predetermined threshold is 5,000 Hertz.

Certain embodiments disclosed herein include any of the devices, computer readable mediums, or systems, noted above or below, wherein each of the plurality of first frequencies is between 5,000 Hertz and 10,000 Hertz.

Certain embodiments disclosed herein include any of the devices, computer readable mediums, or systems, noted above or below, wherein each of the plurality of first frequencies is above 10,000 Hertz.

Certain embodiments disclosed herein include any of the devices, computer readable mediums, or systems, noted above or below, further comprising or being configured to:

Certain embodiments disclosed herein include any of the devices, computer readable mediums, or systems, noted above or below, wherein a width of a pulse of each of the plurality of first frequencies is 200 microseconds.

Certain embodiments disclosed herein include any of the devices, computer readable mediums, or systems, noted above or below, wherein a width of a pulse of each of the plurality of first frequencies is between 50 and 200 microseconds.

Certain embodiments disclosed herein include any of the devices, computer readable mediums, or systems, noted above or below, w wherein a width of a pulse of each of the plurality of first frequencies is less than 100 microseconds.

Certain embodiments disclosed herein include any of the devices, computer readable mediums, or systems, noted above or below, wherein a charge delivered over a time length of each of the plurality of first waveforms is zero.

Certain embodiments disclosed herein include any of the devices, computer readable mediums, or systems, noted above or below, wherein a charge delivered via each of the plurality of first waveforms is asymmetrically balanced.

Certain embodiments disclosed herein include any of the devices, computer readable mediums, or systems, noted above or below, wherein a charge delivered over a time length of the second waveform is zero.

Certain embodiments disclosed herein include any of the devices, computer readable mediums, or systems, noted above or below, wherein a charge delivered via the second waveform is asymmetrically balanced.

Certain embodiments disclosed herein include any of the devices, computer readable mediums, or systems, noted above or below, wherein each of the plurality of first waveforms is a discontinuous waveform, wherein the second waveform is a continuous waveform.

Certain embodiments disclosed herein include any of the devices, computer readable mediums, or systems, noted above or below, wherein each of the plurality of pairs of electrodes has an interelectrode distance, wherein the interelectrode distance of each of the plurality of pairs of electrodes is defined based on a predetermined carotid triangle height.

Certain embodiments disclosed herein include any of the devices, computer readable mediums, or systems, noted above or below, wherein the interelectrode distance of each of the plurality of pairs of electrodes is between 3 and 7 centimeters.

Certain embodiments disclosed herein include any of the devices, computer readable mediums, or systems, noted above or below, wherein each electrode among the plurality of electrode pairs has a diameter between 1 and 3 centimeters.

Certain embodiments disclosed herein include any of the devices noted above or below, further comprising: a resistive sensor adapted to measure resistance of the device; and a switch, wherein the switch is configured to turn on when the resistance measured by the resistive sensor is above a predetermined threshold

Certain embodiments disclosed herein include any of the devices noted above or below, further comprising: an impedance sensor adapted to measure impedance of the device; and a switch, wherein the switch is configured to turn on when the impedance measured by the impedance sensor is within a predetermined range.

Certain embodiments disclosed herein include any of the devices, computer readable mediums, or systems, noted above or below, wherein the controller is further configured to cause the current delivered from the power source to each of the pair of electrodes at a first current level and then to incrementally increase the current from the first current level to a second current level.

Certain embodiments disclosed herein include any of the devices noted above or below, further comprising: an adhesive layer, wherein the adhesive layer is adapted to adhere to a surface of skin.

Certain embodiments disclosed herein include any of the devices, computer readable mediums, or systems, noted above or below, wherein each pair of electrodes is adapted to apply a current delivered from the power source in order to stimulate a vagus nerve of the subject.

Certain embodiments disclosed herein also include a non-transitory computer readable medium having stored thereon causing a processing circuitry to execute a process, the process comprising: sending control signals in order to control current delivered from a power source to a plurality of pairs of electrodes, wherein each pair of electrodes is adapted to apply a current delivered from the power source in a respective current path of the plurality of current paths in order to stimulate a vagus nerve of a subject, wherein the plurality of current paths have a plurality of first waveforms with a plurality of first frequencies, wherein the plurality of current paths overlap at an interference point having a second waveform with a second frequency, wherein the control signals are sent such that each of the plurality of first frequencies is higher than the second frequency.

Certain embodiments disclosed herein also include a system for interferential current stimulation. The system comprises: a processing circuitry; and a memory, the memory containing instructions that, when executed by the processing circuitry, configure the system to: a processing circuitry; and a memory, the memory containing instructions that, when executed by the processing circuitry, configure the system to: send control signals in order to control current delivered from a power source to a plurality of pairs of electrodes, wherein each pair of electrodes is adapted to apply a current delivered from the power source in a respective current path of the plurality of current paths in order to stimulate a vagus nerve of a subject, wherein the plurality of current paths have a plurality of first waveforms with a plurality of first frequencies, wherein the plurality of current paths overlap at an interference point having a second waveform with a second frequency, wherein the control signals are sent such that each of the plurality of first frequencies is higher than the second frequency.

### BRIEF DESCRIPTION OF THE DRAWINGS

The subject matter disclosed herein is particularly pointed out and distinctly claimed in the claims at the conclusion of the specification. The foregoing and other objects, features, and advantages of the disclosed embodiments will be apparent from the following detailed description taken in conjunction with the accompanying drawings.

FIG. 1 is a diagram illustrating a device placed on a carotid triangle according to an embodiment.

FIG. 2 is mechanism of action diagram illustrating vagus nerve stimulation utilized to describe various disclosed embodiments.

FIG. 3 is a schematic diagram illustrating components of a device in accordance with various disclosed embodiments.

FIG. 4 is a diagram illustrating a dumbbell-shaped design of a device according to an embodiment.

FIG. 5 is a diagram illustrating a necklace-shaped design of a device according to an embodiment.

FIG. 6 is a wire diagram illustrating connections between terminals and electrodes according to an embodiment.

FIG. 7 is a diagram illustrating a bandage-shaped design of a device according to an embodiment.

FIG. 8 is a schematic diagram of electrical components in accordance with various disclosed embodiments.

FIG. 9 is a system diagram utilized to describe various disclosed embodiments.

FIG. 10 is a hardware architecture diagram utilized to describe various disclosed embodiments.

FIG. 11 is a top exploded view illustrating components of a device according to an embodiment.

FIG. 12 is a bottom exploded view illustrating components of a device according to an embodiment.

FIG. 13 is an assembled view of a device according to an embodiment.

FIG. 14 is an exploded view of a housing according to an embodiment.

FIG. 15 is an illustration of a device placed on a user.

FIG. 16 is a schematic diagram illustrating a device design utilizing multiple pairs of electrodes according to an embodiment.

FIG. 17 is a waveform diagram illustrating current waveforms delivered across two electrode pairs which may be utilized in accordance with certain embodiments.

FIG. 18 is a waveform diagram illustrating current waveforms delivered across four electrode pairs which may be utilized in accordance with certain embodiments.

FIG. 19 is a waveform diagram illustrating a summed biphasic waveform at an interference point.

FIG. 20 is a schematic diagram of a hardware layer of computing components of a device according to an embodiment.

FIG. 21 is a flowchart illustrating a method for vagus nerve stimulation according to an embodiment.

### DETAILED DESCRIPTION

It has been identified that senses of a subject such as vision, hearing, and touch, are more accurate when the subject is attentive and alert. When highly attentive, the brain activates neural circuitry in ways that optimize how sensory information is encoded and processed. This optimized sensory processing removes noise and may increase the accuracy and detail of sensory information, which allows for more accurate perception. Accordingly, various disclosed embodiments allow a subject (e.g., a subject wearing a wearable device) to activate this neural circuitry on demand in order to enhance sensory acuity and reduce misperceptions.

In this regard, it has been identified that, after sensory information is encoded by receptors (e.g., eyes, ears, skin), the sensory information enters the brain as a pattern of neural activity encoded as a neural signal. This signal is then processed through multiple brain regions before it is perceived during sensory processing, which lets brain regions along this pathway such as, but not limited to, the thalamus, to influence how the world is perceived. Perceptual acuity is therefore dependent upon high-fidelity, accurate sensory processing. As thalamic processing is a biological process, suboptimal conditions (e.g., brain states, neurochemical levels) may introduce noise or dysfunction in neural circuits that degrades the sensory information processed, thereby resulting in degraded acuity of perceived sensory content.

It has further been identified, via testing on subjects such as rats, that steady activation of the locus coeruleus-norepinephrine (LC-NE) system can cause a steady increase in NE concentration in the thalamus that enables optimization of intrathalamic dynamics for more accurate and detailed sensory processing. It has been identified that such an NE-enhanced sensory processing state can translate to enhanced sensory acuity as evidenced by improved performance on sensory discrimination tests. Moreover, it is noted that the function of neuromodulatory systems are similar in humans and rodents such that it has been identified that the applications of these findings (including the various disclosed embodiments) may be applied to humans and potentially other animals as well.

On a related note, it has also been identified that the increased NE levels used to improve sensory processing alter the response properties of thalamic neurons encoding sensory information in a manner that reduces the influence of calcium t-channels (membrane channels in neurons that influence electrical potential and therefore spiking response) responsible for burst firing. It has been identified that burst firing, and influence of calcium t-channels, can impair the ability of neurons to accurately encode details of sensory stimuli such that the process of burst firing tends to decrease sensory acuity.

Further, it has been identified that direct activation of the LC may be dangerous in humans due to the LC's location deep in the brainstem near regions involved with regulation of the autonomic nervous system that controls critical involuntary functions. However, it has been identified that vagus nerve stimulation (VNS) can be utilized to activate the LC. The vagus nerve innervates the nucleus tractus solitarius and provides excitatory input. When VNS is applied, the nucleus of the solitary tract (NTS) is activated, which in turn innervates and activates the LC. Moreover, it has been further identified that certain patterns of VNS particularly demonstrate beneficial effects on sensory processing similar to that seen with LC stimulation such that these patterns may be leveraged, in accordance with various disclosed embodiments, in order to achieve activation of the LC-NE system to produce comparable or otherwise similar effects to direct activation of the LC, thereby allowing for utilizing LC activation to improve sensory processing while avoiding certain harmful effects of implants or energy targeted for direct LC activation.

In this regard, it has further been identified that certain VNS patterns may be utilized in order to provide a benefit to sensory processing, Some such stimulation patterns may include fully continuous electrical waveforms with no quiescent periods (e.g., continuous alternating current), and other such stimulation patterns may include transient stimulation events with quiescent intervals in between (e.g., pulsed stimulation with quiescent inter-pulse-intervals). Further, either type of those waveforms can be either delivered nonstop or can be delivered with a duty-cycle stimulation pattern (i.e., a stimulation pattern including multiple pulses or a continuous waveform) being repeatedly delivered for some time period (on cycle) and then not delivered for another time period (off cycle), with this cycle repeating continuously. Stimulation patterns can also be modulated over time by changing frequency or amplitude of current.

In this regard, it has been identified that stimulation patterns which have duty cycles that create quiescent periods greater than about 10 seconds tend to create a fluctuating influence on NE levels in the brain and, in turn, a fluctuating influence on sensory processing which may yield suboptimal results. It has further been identified that stimulation patterns which contain quiescent periods longer than about 100 milliseconds may result in a continuous sensation of electrical current at the skin. In at least some implementations, such a continuous sensation of electrical current may be desirable to avoid, for example, to avoid potential distractions caused by such stimulation. It has yet further been identified that a stimulation pattern with less than about 40 ms of quiescent time between stimulation pulses may result in desensitization of the skin within about 5 minutes of use, with some subjects experiencing desensitization within 1 minute and others experiencing desensitization up to 10 minutes after stimulation begins. This may allow for reducing any sensation at the interface site between the skin and the stimulating device producing the transcutaneous electrical field. Therefore, an uninterrupted tonic stimulation pattern may provide better results for at least some implementations. More specifically, an uninterrupted tonic stimulation pattern may mimic the continuous tonic activation of the LC-NE system that occurs naturally during increased attention or arousal, and may therefore improve effects on sensory perception.

It has also been identified that the LC-NE system may modulate sensory processing of visual and auditory modalities in a similar manner. More specifically, it has been identified that increased attention and NE levels correlated with reduced bursting activity in neurons processing visual and auditory information (e.g., in thalamus, cortex, etc.). Accordingly, it has been identified that increases in forebrain NE concentration, and suppression of calcium t-channel bursting activity, may be utilized to enhance thalamic information processing and transmission. It has therefore been determined that the NE system may be able to optimize sensory perception.

To this end, various disclosed embodiments leverage these discoveries regarding the effects of LC-NE activation on sensory processing and the use of VNS to activate the LC in a manner which improves safety as compared to, for example, direct LC stimulation. At least some disclosed embodiments utilize patterns of VNS stimulation which have been discovered to yield particularly optimal sensory processing improvements. In particular, various disclosed embodiments utilize an uninterrupted tonic pattern of VNS which has been identified to allow for enhanced sensory processing.

In particular, various disclosed embodiments may use or include an externally worn, transcutaneous cervical vagus nerve stimulation (nVNS) patch. Such a patch may be conveniently removable in order to allow users to utilize the patch during specific times, for example, when participating in sports, when operating in dangerous conditions, or otherwise when it is desirable to improve sensory processing. This patch may also, in accordance with various disclosed embodiments, be designed to be fully noninvasive and to deliver electrical current to the vagus nerve through the skin via flat electrodes which may rest above where the vagus nerve runs through the neck. This patch may also have an adhesive layer which holds the device and electrodes in contact with the skin. Accordingly, the disclosed nVNS techniques and devices allow for providing a safe and effective means of inducing neuromodulation which can be activated and deactivated on-demand.

The disclosed embodiments may therefore be utilized in different implementations which may require sensory processing improvements or otherwise for which sensory processing improvements may be desirable. In particular, various disclosed embodiments may aid individuals with neurodegenerative disorders which affect sensory processing such as, but not limited to, Alzheimer's disease, Parkinson's, age-related sensory loss, shift-work syndrome, insomnia, and the like. More specifically, various disclosed embodiments may be utilized in order to treat patients with these impairments. Additionally, individuals with disorders such as ADHD and dyslexia may be treated in order to mitigate the effects of these disorders on sensory processing.

In addition to treating individuals having certain disorders, various disclosed embodiments may have applicability in contexts such as sports, gaming, or other contexts where enhanced sensory processing might be desirable including, but not limited to, students, artists, individuals enjoying sensorial content, military, and the like. As a non-limiting example, athletes playing a physical sport or e-sport may benefit from improved sensory processing which may allow them to more accurately perceive visual, auditory, and tactile feedback. This, in turn, may reduce misperceptions, miscommunications, clumsy hands, combinations thereof, and the like, that could impair performance.

Likewise, individuals performing tasks which are inherently dangerous and require sharp sensory processing (as misperceptions may result in costly human error) may benefit from various disclosed embodiments.

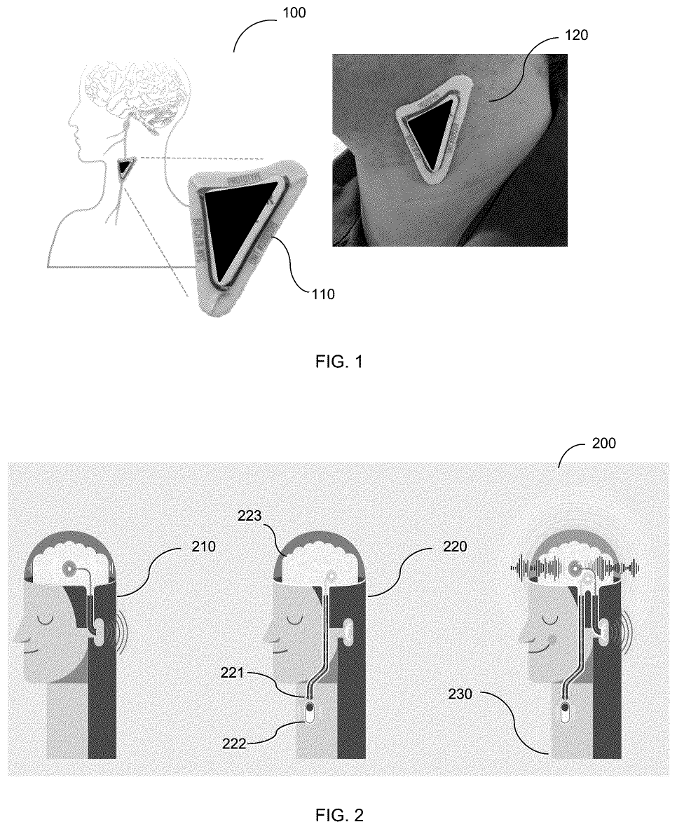

FIG. 1 is a diagram 100 illustrating a device placed on a carotid triangle according to an embodiment. The diagram 100 illustrates a device 110 in the form of a triangular patch which may be disposed on a neck 120 of a user in accordance with various disclosed embodiments. More specifically, in an embodiment, the device 110 is aligned above the carotid artery of the neck because the vagus nerve runs adjacent and parallel with the carotid artery underneath the carotid triangle of the neck. In accordance with various disclosed embodiments, the device 110 is disposed on a carotid triangle of the neck 120 of the user. In the embodiment depicted in FIG. 1, the device 110 may be placed on a user non-invasively, i.e., without needing to pierce the skin of the neck 120 of the user or otherwise implanting the device 110 underneath the skin of the neck 120. Thus, the disclosed embodiments may allow for improving sensory processing non-invasively.

FIG. 2 is a mechanism of action diagram 200 illustrating vagus nerve stimulation utilized to describe various disclosed embodiments. The diagram 200 illustrates certain aspects in accordance with various disclosed embodiments. More specifically, the diagram 200 illustrates conversion 210 of sound into a neural signal by the ear, which is then processed by the brain before perception occurs. The diagram 200 also illustrates stimulation 220 of a vagus nerve 221 via a device 222 and demonstrates how stimulation of the vagus nerve 221 causes norepinephrine levels to increase in regions of a brain 223 which process auditory information. The device 222 may be a device configured in accordance with one or more disclosed embodiments. Further illustrated are effects 230 of the resulting increased norepinephrine levels resulting in enhanced sensory processing, which in turn results in a clearer perception of the sound.

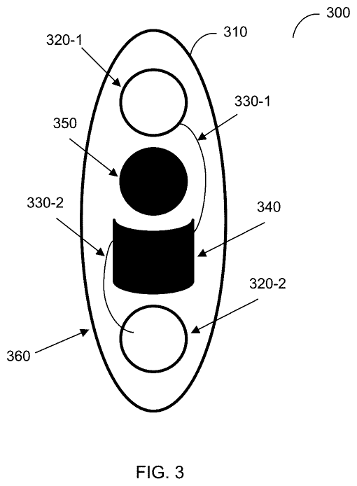

In accordance with at least some disclosed embodiments, stimulation may be targeted at a section of the vagus nerve located under the carotid triangle. To this end, in an embodiment, the device 222 is disposed on a left side of the neck. As discussed further below with respect to FIG. 3, the device 222 may include a pair of electrodes (e.g., hydrogel electrodes) to facilitate delivery of electric energy targeting the vagus nerve 221 as it runs under the location of the carotid triangle. In at least some embodiments, the device 222 does not extend above the neck onto the jaw.

In accordance with various disclosed embodiments, the device 222 utilizes directed energy targeted at certain tissues (e.g., neural, organ, muscles, etc.). The directed energy may be delivered with specific patterns (e.g., waveform, intensity, frequency, etc.) in order to evoke certain changes in neural activity, behavior, and cognition. To this end, the device 222 may be realized as, for example but not limited to, a neural stimulation patch adapted to activate neural circuitry (e.g., the norepinephrine system via stimulation of the vagus nerve 221) that causes enhancement of central sensory processing. Specifically, the enhanced central sensory processing may improve acuity of any of vision, hearing, touch, and the like. In at least some implementations, the enhanced sensory processing may be realized instantly after stimulation begins and may continue until the device 22 is removed. As noted above, vagus nerve stimulation may allow for enhancing sensory processing while minimizing or avoiding side effects.

In at least some embodiments, the device 222 may be realized as or using an adhesive patch. In some implementations, the entire patch (i.e., the entire device 222) may be disposable. In other implementations, some components of the device 222 (e.g., a microchip, not shown in FIG. 2) are durable and components such as electrodes and batteries are replaceable. In an embodiment, the device 222 has dimensions between 5 to 6 centimeters tall, 2 to 3 centimeters wide, and thickness less than 0.5 centimeters.

FIG. 3 is a schematic diagram illustrating components of a device 300 in accordance with various disclosed embodiments.

As depicted in FIG. 3, an outer edge 310 of the device 300 may be designed with a shape that may vary on the use case. The example shape of the outer edge 310 depicted in FIG. 3 may allow for minimizing the size of the device 310, but other shapes may be equally utilized in accordance with various disclosed embodiments. For example, in some embodiments, the device 300 may be triangular as depicted in FIG. 1. Such a triangular shape may aid a user in correctly aligning placement of the device 300 with respect to a carotid triangle. The outer edge 310 may be, but is not limited to, around 0.5 centimeters thick.

The device 300 includes a pair of electrodes 320-1 and 320-2. In an embodiment, each of the electrodes 320-1 and 320-2 is a gel electrode. In a further embodiment, each electrode 320-1 and 320-2 has a diameter of 2 centimeters. In yet a further embodiment, a distance between center points of the electrodes 320-1 and 320-2 is 4 centimeters. In another embodiment, the distance between such center points is between 4 and 5 centimeters.

In this regard, it is noted that optimal interelectrode distance in at least some embodiments is based on the height of the carotid triangle of a given user. More specifically, electrodes which are placed outside of the carotid triangle may face challenges in optimally penetrating the vagus nerve with current due to increased muscle between the skin and the target nerve at locations outside of the carotid triangle. It has been identified that a distance between 4 and 5 centimeters between electrodes may allow for optimizing penetration of current in accordance with various disclosed embodiments. Moreover the shape of the electrodes may be selected in order to maximize surface area, distance between center points, or both.

The electrodes 320-1 and 320-2 are connected via respective wires 330-1 and 330-2 to a circuit board 340 such as, but not limited to, a printed circuit board. As depicted in FIG. 3, the circuit board 340 is shaped in order to partially surround a battery 350, although other shapes may be equally utilized in at least some other embodiments. In some implementations, the circuit board 340 and the battery 350 may be stacked on top of each other. In an embodiment, the circuit board has a surface area of around 2 square centimeters. In a further embodiment, the circuit board is 1 millimeter thick. In yet a further embodiment, the circuit board is opaque. In yet a further embodiment, the circuit board is transparent. In yet a further embodiment, the circuit board is flexible. In some embodiments, the electrodes 320-1 and 320-2 are surrounded by an edge 360 of the device 300 which extends at least 1 centimeter beyond the outer boundary of the electrodes 320-1 and 320-2.

In this regard, it has been identified that maintaining a minimal surface area for the electrodes may be utilized to ensure that the current density during stimulation does not increase to a level where discomfort or injury would occur. It has further been identified that a surface area of around at least 2 square centimeters allows for delivering a sufficient current to enhance sensory processing via vagus nerve stimulation while minimizing or avoiding any discomfort or injury. In other embodiments, electrodes with larger surface areas may be utilized. In that regard, it is noted that larger electrodes may allow for minimizing cutaneous pain due to reduced current density at skin, and may therefore be particularly suitable for deeper nerve stimulation and/or constant recruitment.

It has further been identified that conductive hydrogel electrode coatings may be utilized to improve the performance of bionic devices, which may utilize lower amounts of energy than certain metal electrodes as well as which may exhibit better biocompatibility and adhesive properties. Thus, in at least some embodiments, hydrogel electrodes are utilized. In other embodiments, other materials with suitable conductivity to provide vagus nerve stimulation in order to realize enhanced sensory processing may be utilized (e.g., carbon/rubber, silver/silver chloride, stainless steel, nickel, gold, etc.).

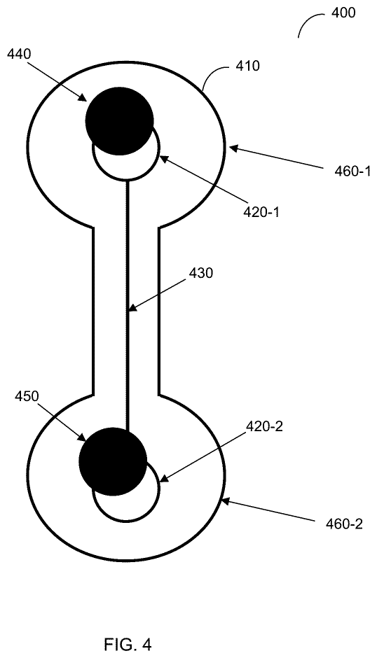

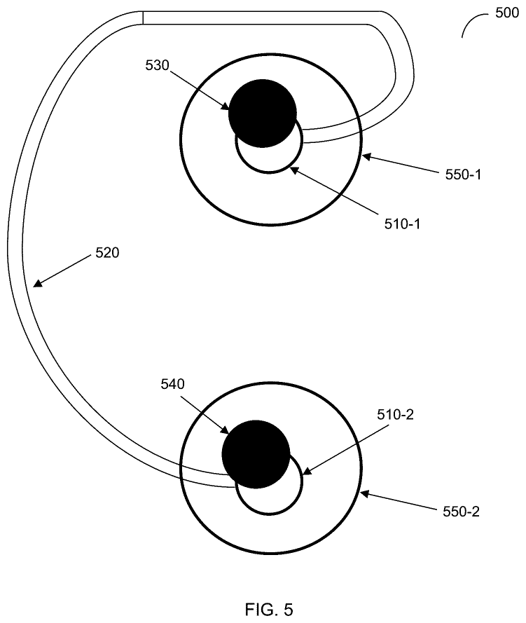

The wires 330-1 and 330-2 may be or may include, but are not limited to, conductive wires, traces, and the like. In some embodiments, wires may not be utilized for components which are connected directly (e.g., via solder contact points). Examples of such direct connections are depicted in FIGS. 4 and 5, where electrodes are connected directly to circuit boards and batteries, respectively.

The battery 350 is connected to the circuit board 340, either via direct contact or via a wire (not shown in FIG. 3). To this end, the battery 350 may be stacked on top of the circuit board 340 (not shown) or placed side-by-side (as shown). In an embodiment, the battery has a diameter of about 2 centimeters and a thickness of about 1 millimeter. In a further embodiment, the battery is opaque. In an example implementation, the battery may be a coin cell battery (e.g., lithium ion). In yet a further embodiment, the battery may be transparent. In yet a further embodiment, the battery may be flexible.

The circuit board 340 is adapted to convert a voltage differential from the battery 350 into pulsed waveform patterns as described herein. In an embodiment, a timer integrated circuit is utilized for pulse generation. In another embodiment, an operational amplifier is used for current delivery. In some implementations, a microcontroller may be used; in other implementations, a less complicated (and therefore less power consuming) chip may be utilized (e.g., an application-specific integrated circuit). In some implementations, an aperture in a chip (not shown) of the circuit board 340 may be defined in order to allow for placement of the battery therein.

The various components shown in FIG. 3 may be encased or otherwise enclosed in a film (not depicted in FIG. 3) used to realize the device 300 as a patch. In some implementations, the film may be transparent in order to allow natural skin color to show through the film. Such a film may be around 0.2 millimeters thick. Moreover, the film may be made of materials such as, but not limited to, silicone, plastic, resin, epoxy, thin-film laminates, metals, sprayable conformal coatings (e.g., parylene, silicone dioxide, polytetrafluoroethylene, acrylic, epoxy, etc.) combinations thereof, and the like. The film, in at least some embodiments, may be used to form a water-resistant enclosure. In other embodiments a water-resistant film is applied directly to all electrical sub-components.

FIG. 4 is a diagram illustrating a dumbbell-shaped design of a device 400 according to an embodiment.

As depicted in FIG. 4, a pair of electrodes 420-1 and 420-2 are connected via a wire 430. In the embodiment shown in FIG. 4, a circuit board 440 is stacked on top of one of the electrodes 420-1 and a battery 450 is stacked on top of another of the electrodes 420-2. Such a stacking design may reduce the area of the device 400 that is opaque (e.g., allowing more space to be occupied by a transparent film) in exchange for increased thickness of the device 400.

In an embodiment, the electrodes 420-1 and 420-2 each have a diameter of 2 centimeters and a thickness of 1 millimeter. The circuit board 440 may be opaque and, in an embodiment, has a surface area equal to or greater than 3 centimeters. The battery 450 maybe opaque and, in an embodiment, have a thickness of 1.6 millimeters and a diameter of 2 centimeters. In some embodiments, the electrodes 420-1 and 420-2 are surrounded by edges 460-1 and 460-2, respectively, which extend at least 1 centimeter beyond the outer boundary of the respective electrodes 420-1 and 420-2.

FIG. 5 is a diagram illustrating a necklace-shaped design of a device 500 according to an embodiment. In this regard, it is noted that a device 500 which wraps around a user's neck may be desirable in order to demonstrate a natural-looking appearance like neckwear such as necklaces or scarves, or to be covered by such neckwear. The embodiment depicted in FIG. 5 may provide such a natural-looking or convenient to cover appearance.

As depicted in FIG. 5, electrodes 510-1 and 510-2 are connected via a wire 520 designed to wrap around a neck (not shown in FIG. 5). To this end, the wire 520 may be long enough (e.g., between 10 and 20 inches) in order to allow the wire to wrap around a user's neck. Moreover, in some implementations, the wire may be made of metal in order to appear more like a necklace. A circuit board 530 and a battery 540 may be stacked on top of the electrodes 510-1 and 510-2, respectively.

In an embodiment, the electrodes 510-1 and 510-2 each have a diameter of 2 centimeters and a thickness of 1 millimeter. The circuit board 530 may be opaque and, in an embodiment, has a surface area equal to or greater than 3 centimeters. The battery 540 maybe opaque and, in an embodiment, have a thickness of 1.6 millimeters and a diameter of 2 centimeters. The electrodes 510-1 and 510-2 may be surrounded by edges 550-1 and 550-2, respectively, which extend at least 1 centimeter beyond the outer boundary of the respective electrodes 510-1 and 510-2.

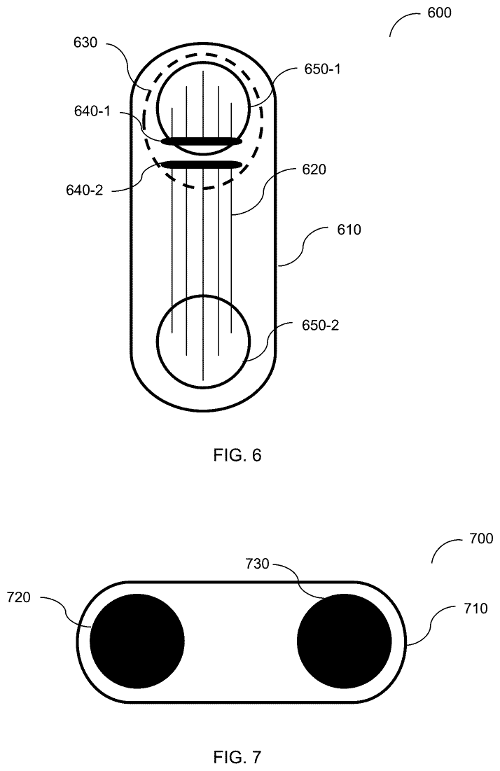

In some embodiments, wiring between a microchip and electrodes is used to create a pattern within a film section of a patch including a device. In an embodiment, the pattern is a trace pattern. An example wire diagram illustrating such a trace pattern is now described with respect to FIG. 6. In some embodiments, the wire traces are embedded in the thin film.

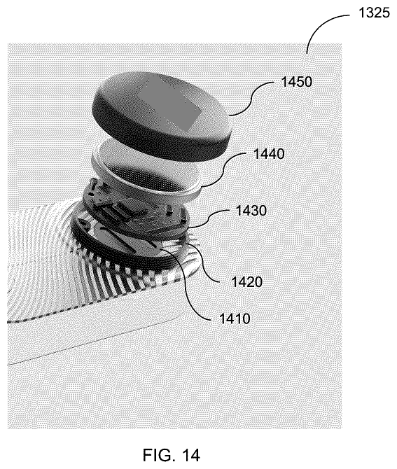

FIG. 6 is a wire diagram 600 illustrating connections between terminals and electrodes according to an embodiment. As depicted in FIG. 6, a device 610 has a set of wires 620 forming a trace pattern. The wire diagram 600 further indicates a location 630 upon which a housing (not shown) may be placed. Such a housing may include, but is not limited to, a circuit board and a battery. Non-limiting example housing components are described further below with respect to FIG. 14. In some embodiments, a microphone (not shown) may also be included.

The wire diagram 600 further illustrates a set of connection terminals 640-1 and 640-2 which may be utilized to electrically connect to a microchip disposed in the housing (not shown) placed upon the location 630. Such a microchip may have two separate outputs which may connect to two distinct respective electrodes 650-1 and 650-2. In some embodiments, a plurality of distinct outputs and electrode contacts points can be used. Multiple electrodes allow for current to flow inwards from one electrode into the tissue and outwards from the tissue into the other electrode. In a further embodiment, one of the electrodes (e.g., the electrode 650-1) only connects to one terminal (e.g., the terminal 640-1), while the other electrode (e.g., the electrode 650-2) only connects to another terminal (e.g., the terminal 640-2).

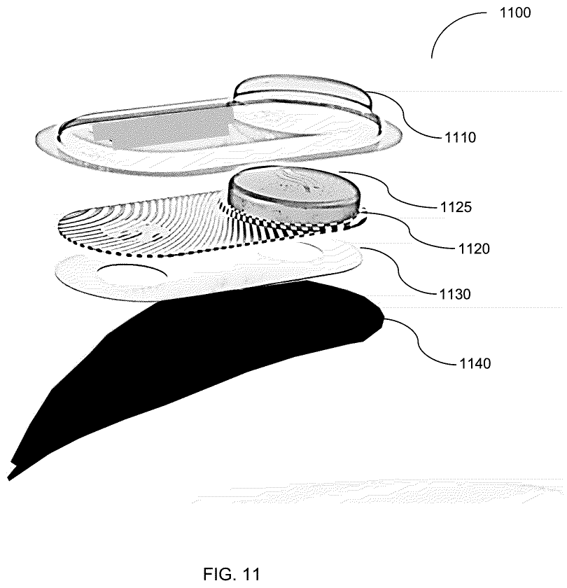

FIG. 7 is a diagram illustrating a bandage-shaped design of a patch 700 according to an embodiment. As depicted in FIG. 7, the patch 700 includes a surface 710 with two electrodes 720 and 730 disposed thereon. The surface 710 may be made of one or more layers such as, but not limited to, an adhesive layer. In some embodiments, the adhesive layer is located on the entire surface 710 of the patch 700 except for portions of the surface 710 where the electrodes 720 and 730 are disposed. In some embodiments, the edges of the patch 700 are rounded in order to improve comfort at skin interface. An example set of layers which may be utilized to realize the surface 710 is discussed further below with respect to FIG. 11.

It should be noted that the various embodiments discussed with respect to FIGS. 3-7 are described with respect to two electrodes, but that additional electrodes may be utilized in accordance with at least some disclosed embodiments. Moreover, the electrodes depicted in FIGS. 3-7 are circular in shape, but that other shapes may be equally utilized without departing from the scope of the disclosure.

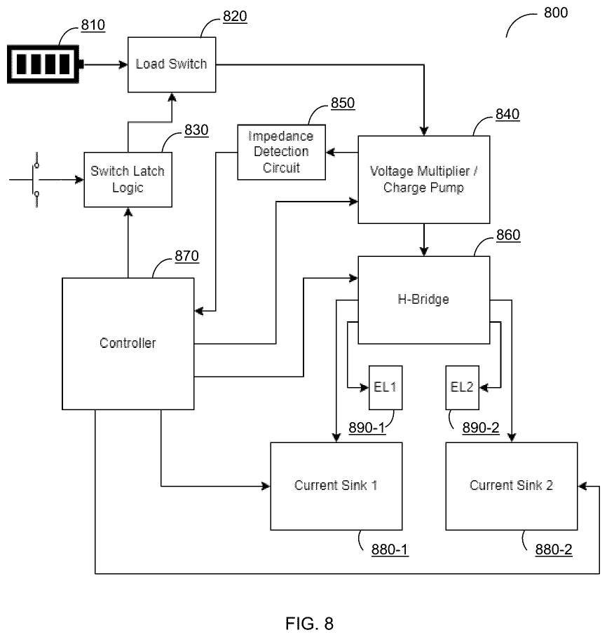

FIG. 8 is a schematic diagram 800 of electrical components in accordance with various disclosed embodiments. The diagram 800 illustrates an example system topology.

The example system topology illustrated in FIG. 8 may be realized on or otherwise via a printed circuit board (PCB, not shown in FIG. 8). In some non-limiting implementations, the PCB may be a rigid type PCB. In other implementations, a flex type PCB may be utilized.

As depicted in FIG. 8, an electrical source 810 such as a battery is electrically connected to a load switch 820, which in turn is communicatively connected to switch latch logic 830 and to a voltage multiplier 840. The voltage multiplier 840 may be, but is not limited to, a Dickson charge pump, and may be utilized for a current sink supply. The voltage multiplier 840 is connected to an impedance detection circuit 850 and a H-bridge 860.

In at least some implementations, the electrical source 810 is adapted such that it is capable of powering a device as described herein for at least 8 hours of continuous use. In a further implementation, the electrical source 810 is adapted to enable at least 12 hours of continuous device use. The electrical source 810 may be, but is not limited to, a non-rechargeable battery, a durable rechargeable battery, and the like. Non-limiting example types of battery which may be utilized in accordance with various implementations include alkaline, lithium-ion, lead-acid, nickel-cadmium, nickel-metal hydride, lithium polymer, zinc-Carbone, silver oxide, zinc-air, and the like.

In an embodiment, the electrical source 810 is adapted such that a device including the electrical source 810 is adapted to apply a continuous stimulation pattern for a time period between 1 and 12 hours. In a further embodiment, the device is adapted such that a maximum length of time of quiescence is 10 seconds during the time period in which the continuous stimulation pattern is applied. That is, the maximum duration of any period of inactive time (i.e., time in which pulses are not being emitted) during the stimulation is 10 seconds. In another embodiment, the device is adapted such that a maximum length of time of quiescence is 100 milliseconds during the time period in which the continuous stimulation pattern is applied.

Outputs of the impedance detection circuit 850 are input to a controller 870, which in turn sends control signals to the switch latch logic 830, the voltage multiplier 840, the H-bridge 860, a first current sink 880-1, and a second current sink 880-2. An additional charge pump (not shown) may be integrated into a chip (not shown) of the controller 870 in order to provide a target voltage output such as, but not limited to, an output of about 9 volts with a maximum current amplitude of around 10 milliamps. The H-bridge 860 may deliver power to a pair of electrodes 890-1 and 890-2.

In an embodiment, the system topology shown in FIG. 8 is utilized to deliver an output current pulse having an amplitude between 4 and 12 Milliamps (mA), a square bi-polar (biphasic) waveform, having two phases each with a width of 100 microseconds per phase (for a 200 microsecond long total pulse), and a repetition frequency of 30 Hertz (Hz). In some embodiments, the frequency is between 15 and 90 Hz. The square biphasic waveform may include multiple energy pulses having the two phases. In some embodiments, the width per phase is between 50 and 500 microseconds. In some embodiments, the phases have different widths.

That is, the system topology may be utilized to repeatedly deliver stimulation pulses. These pulses have an induced change in electrical potential between two points (e.g., points of electrodes as discussed herein), which causes current to flow through the tissue between the electrodes 890-1 and 890-2. In some embodiments, the pulses are biphasic pulses which alternate the current direction at some point during the pulse (which prevents charge build up in the tissue and improves activation of neural targets). In a further embodiment, the amplitude may be configurable to different predetermined settings. In yet a further embodiment, the predetermined settings include 5 mA, 7 mA, and 9 mA. In another embodiment, the current pulse which is delivered is between 7 to 12 mA. Alternatively, the current pulse which is delivered is less than or equal to 60 mA. It has been discovered that 60 mA is likely a top end of a safe range of potential amplitude values. It has also been identified that 7 to 12 mA may provide optimal stimulation conditions for at least some disclosed embodiments. It has been identified that such ranges tend to be comfortably tolerated by subjects with normal body mass index. Higher current levels (up to 60 mA) may be used in at least some embodiments in order to properly reach and engage the vagus nerve in targets with relatively more deep vagus nerve locations relative to skin surface (e.g., obese users).

In some embodiments, a time of around 0.01 milliseconds (ms) is inserted between phases of the biphasic pulse in order to produce an interphase gap. In this regard, it has been identified that such an interphase gap has demonstrated effectiveness in improving activation of target neural structures, which in turn can further enhance sensory processing, minimize or avoid side effects of nerve stimulation, or both, in accordance with various disclosed embodiments.

In another embodiment, an asymmetric biphasic waveform is used. More specifically, in a further embodiment, a second phase of the biphasic waveform is between 10 and 30 percent weaker than a first phase (e.g., weaker as measured in current amps, i.e., 10 to 30 percent lower current amps). In this regard, it is identified that symmetric biphasic waves can, in at least some instances, cause depolarization from charge build up over time because the potential within the tissue is affected slightly differently by the first and second phase due to the effects of the first phase slightly influencing the effects of the second phase (e.g., during extended use). Accordingly, using a slightly asymmetric biphasic waveform such as a waveform where the second phase is 10 to 30 percent weaker than the first phase may avoid or otherwise mitigate depolarization.

In some embodiments, electrical current is provided according to a ramp-up procedure. In such a ramp-up procedure, the full amplitude current of the stimulation pattern to be applied is not provided initially when the device or patch is applied to the skin. In this regard, it has been identified that a sudden change from no stimulation to full strength stimulation (e.g., at a rate of over 10 mA/second) may be shocking or uncomfortable for the user. To this end, in some further embodiments, amplitude may be increased in incremental steps (e.g., increasing by 0.05 mA/second). In a further embodiment, a ramp-up pace between 1 and 10 mA/second is utilized to increase the amplitude in incremental steps. In yet a further embodiment, a ramp-up pace between 1 and 5 mA/second is utilized. In this regard, it has been discovered that a ramp-up pace between 1 and 5 mA/second tends to provide the least amount of discomfort.

In a further embodiment, the ramp-up procedure begins with an immediate increase in stimulation to a first level, and then incrementally increasing the amplitude to a second level (i.e., the full amplitude current to be delivered) in multiple increments. In some embodiments, a current value between 1 and 5 mA is used as the first level to be applied immediately upon stimulation beginning. In a further embodiment, a current value of 5 mA is used as the first level, and a current value between 7 and 12 mA is used as the second level (full amplitude current). Alternatively, the second level may be any value up to 60 mA.

In this regard, it has been discovered that a slow ramp-up to the full amplitude current tends to cause more discomfort for users than an immediate jump to a lower level of current followed by a ramp-up to the full level of current. It has further been discovered that current values between 1 and 5 mA are the lowest perceptible current for most individuals using the stimulation pattern as described herein. It has also been identified that some individuals exhibit more discomfort for current values between 1 and 5 mA than for a current value of 5 mA such that, for some implementations, it may be desirable to jump immediately to 5 mA to minimize discomfort. It has further been discovered that maximum current values between 7 and 12 mA have been found to provide adequate stimulation for many use cases without causing significant levels of discomfort such that current values between 7 and 12 mA may be suitable full amplitude current values. That said, it has been discovered that current values up to 60 mA are safe levels even if some higher current values up to 60 mA may cause additional discomfort. In that regard, it is noted that some implementations and use cases may tolerate additional discomfort in exchange for a higher current (e.g., to further enhance sensory processing). For example, a military use case, a life-or-death situation, or an athlete may tolerate some degree of discomfort for further improved performance.

In an embodiment, the frequency, waveform, or both, of the stimulation pattern may affect the thresholds to be used. For example, certain frequencies or waveforms may be perceptible at current values outside of 1 to 5 mA and, consequently, the first level may be set to a different current value in accordance with certain embodiments and implementations in order to cause initial perception while reducing overall discomfort.

In order to provide a ramp-up as discussed above and to ensure that current is only delivered when product is properly applied to skin, in some embodiments, a circuit designed to deliver transcutaneous stimulation is adapted to measure an impedance value of an attached load and to deliver stimulation when an appropriate load is indicated (e.g., above a predetermined threshold or within a predetermined range of appropriate loads such as, but not limited to, between 500 and 2000 ohms). The ramp-up may be facilitated by adjusting, for example but not limited to, a time course of a charging capacitor which is adapted to create a voltage signal that ramps up over time until it reaches a saturation value and for which the ramp rate properties can be adjusted by varying properties of the capacitor.

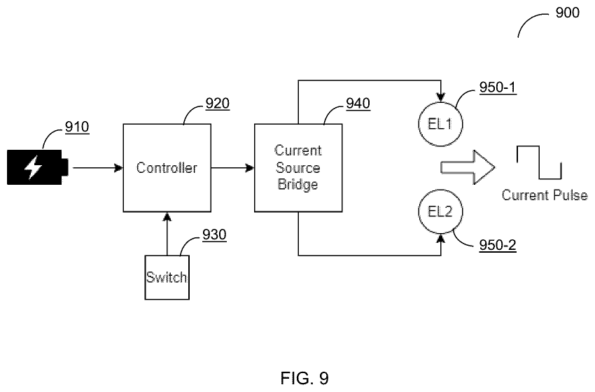

FIG. 9 is a system diagram 900 utilized to describe various disclosed embodiments.

As depicted in FIG. 9, a power source 910 provides power to a controller 920. The controller 920 is activated via a switch 930. The controller 920 sends control signals to a current source bridge 940, which in turn power a pair of electrodes 950-1 and 950-2.

The controller 920 may be realized via one or more chips such as, but not limited to, microchips. As discussed herein, such microchips may be disposed on a printed circuit board (PCB) and enclosed in a housing. The housing may further be designed to create a tight seal in order to prevent water or other fluids from entering the housing and affecting the controller 920. In some embodiments, the chip is enclosed within a film of a patch (not shown) without requiring a housing.

In some embodiments, a physical switch 930 is adapted to keep a battery such as the power source 910 electrically disconnected from the electrodes 950-1 and 950-2 until the battery is removed from packaging (not shown). This may be utilized in order to prevent the battery from draining while in transit and storage. In some embodiments, a digital switch 930 may further be connected to one or more sensors (not shown) configured to measure one or more metrics such as, but not limited to, impedance, current, voltage, resistance, combinations thereof, and the like. Measuring these metrics allows for determining whether any of these metrics are above a threshold, below a threshold, between thresholds, and the like. In such an embodiment where the switch 930 is connected to sensors, the switch 930 is configured to turn on or off depending on whether one or more metrics are above, below, or between, respective thresholds. In this regard, a device incorporating the switch 930 and any such sensors is adapted to monitor metrics such as impedance, current, voltage, or resistance, and to turn on (thereby applying current) or shut down (thereby ceasing application of current). Sensors or other components used to measure such metrics may include, but are not limited to, voltage dividers, a resistor with a known value resistance or impedance, one or more comparator circuits (e.g., circuits configured to compare voltage with a reference voltage in order to output values indicating when one voltage is higher than the other), digital control logic, combinations thereof, and the like.

In particular, in some embodiments, a resistive sensor adapted to measure resistance is connected to the switch 930, and the switch 930 is configured to turn on when the resistance measured by the resistive sensor is above a predetermined threshold. Turning the device on when a certain amount of resistance has been detected may be utilized to effectively determine whether a patch or device as described herein is applied to skin and delivering current as described herein only when the device is applied to skin.

In another embodiment, an impedance sensor adapted to measure impedance across the electrodes 950-1 and 950-2 is connected to the switch 930, and the switch 930 is configured to turn on when the impedance measured by the impedance sensor is above a threshold, below a threshold, or within a range bound by thresholds. In this regard, a device incorporating the switch 930 and such an impedance sensor may be effectively adapted to shut down when an appropriate load is not present as defined with respect to one or more predetermined thresholds (e.g., when the impedance is below a predetermined threshold, above a predetermined threshold, outside of a range, combinations thereof, etc.). In a further embodiment, the range of appropriate loads may be between 400 and 4000 Ohms. In some embodiments, stimulation may resume when the load is once again at an appropriate level (e.g., above a threshold, below a threshold, or within a range).

In this regard, it is noted that a device not being appropriately applied to skin may cause impedance or other metrics to fall outside of safe ranges. In such a case, for example, when electrodes of a device are placed such that they do not fully contact an appropriate surface area of the user's skin, such a lack of full contact may cause any current delivered to the skin to exceed a safe or comfortable amount of current given the surface area to which the current is effectively applied. As a non-limiting example, when the device is implemented in a patch which begins to peel such that one of the electrodes ceases being in contact with the skin, a situation in which the device would send the same amount of current through half of the normal surface area of the skin may occur. This imbalance of current to surface area may cause discomfort or injury such that detecting impedance above a threshold and ceasing stimulation when impedance is above the threshold may avoid discomfort or injury. To this end, in an embodiment, the device may be adapted to cease stimulation when the impedance is above a threshold of 10,000 ohms.

Likewise, impedance below a threshold may indicate that the device has short circuited such that the electrodes are not properly electrically isolated from each other. For example, in an embodiment, a normal impedance range may be between 200 and 4000 ohms such that an impedance below 200 ohms may be indicative of a short circuit. In such a case, the device may shut off when the impedance drops below 200 ohms. Impedance may be measured using components or techniques such as, but not limited to, time-domain reflectometry, frequency domain analysis, Wheatstone bridge, impedance-to-digital conversion chips, combinations thereof, and the like.

Similarly, other safety or proper operation thresholds may include, but are not limited to, overvoltage protection thresholds (e.g., over 24 volts), undervoltage protection thresholds (e.g., less than 1 volt), overcurrent protection thresholds (e.g., greater than 1 milliamp above target current), combinations thereof, and the like.

In some embodiments, the switch 930 may be physically attached to packaging such that the switch 930 is pulled (and therefore activated) as part of removing the device from the packaging. In such an implementation, the switch 930 may be realized as a pull-tab or tear strip switch. In other implementations, the switch 930 may be magnetic and held in an “off” position by a small magnet included in the packaging for the device. When the packaging is removed from the device, the removal of the magnet causes the switch 930 to change to an “on” position. Other types of switches (e.g., optical, capacitive, inductive, etc.) may be utilized without departing from the scope of the disclosure.

In other embodiments, the switch 930 may be connected to a button (not shown) disposed on a device such that, when the button is pressed, the switch 930 may activate stimulation as discussed herein. The button may be realized as, for example but not limited to, a toggle, rocker, push, slide, tactile button, capacitive button, physical switch, combination thereof, and the like. In a further embodiment, pressing the button while current is being delivered (i.e., while stimulation is active) may cause the stimulation to deactivate and current to cease being delivered. Some such embodiments may utilize multiple switches or otherwise allow for both activating/deactivating the device via press of a button and activating or deactivating the device when one or more electrical metrics (e.g., voltage, current, impedance, resistance, etc.) meet a threshold or fall within a range as discussed above.

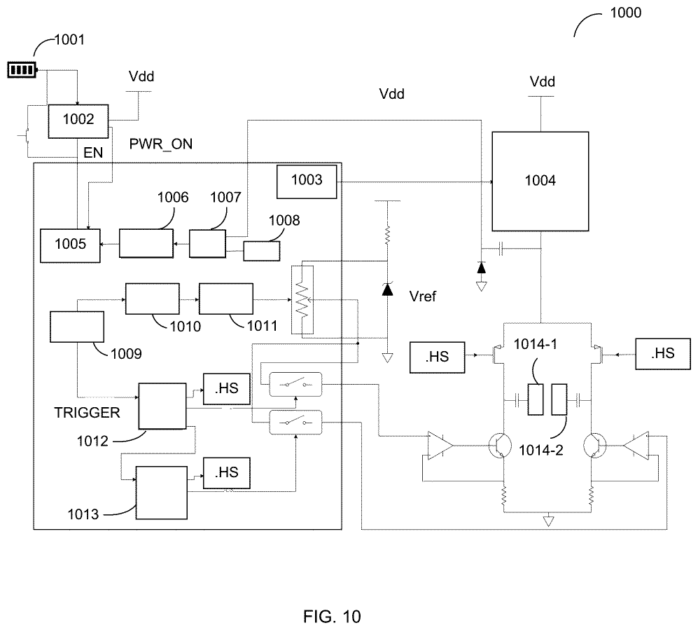

FIG. 10 is a hardware architecture diagram 1000 utilized to describe various disclosed embodiments. The hardware architecture diagram illustrates connections and flows between components including a power source 1001, a load switch 1002, a first clock (e.g., a 1 Kilohertz CLK) 1003, a voltage multiplier or charge pump 1004, a circuit (e.g., a S-R latch 1005), a watch-dog logic component 1006, a comparator 1007, a threshold 1008, a second clock (e.g., a 30 Hertz CLK) 1009, a third clock (e.g., a 1 Hertz CLK) 1010, a ramp logic component 1011, a first pulse generator (e.g., a 100 microsecond pulse generator) 1012, a second pulse generator (e.g., a 100 microsecond pulse generator) 1013, and a pair of electrodes 1014-1 and 1014-2. The ramp logic component 1011 is designed to control ramping up of current, for example as discussed further above.

FIG. 11 is a top exploded view illustrating components of a patch 1100 according to an embodiment. The top exploded view illustrates components including a top cover 1110, a housing 1125 disposed on an electrode apparatus 1120, a adhesive layer of the adhesive layer 1130 of the patch 1100, and a bottom layer 1140. Specifically, FIG. 11 illustrates an assembled version of the patch 1100 including packaging components in the form of a top cover 1110 and a bottom layer 1140.

The top cover 1110 depicted in FIG. 11 is made of a plastic material which may be utilized to protect the patch 1100, for example, during shipping. As discussed above, in some implementations, a film (not shown) may be utilized as a top layer instead of the top cover 1110 depicted in FIG. 11.

The housing 1125 is disposed on the electrode apparatus 1120 and may house components such as, but not limited to, electrodes, wires, circuit boards, and the like. Example components which may be housed in the housing 1125 are described further below with respect to FIG. 14.

The adhesive layer 1130 may be glue or another adhesive used to secure the electrode apparatus 1120 to the skin of the user. The top cover 1110, in order to seal the electrode apparatus 1120 and housing 1125 within a sealed chamber, creates a chamber formed between the top cover 1110 and the bottom layer 1140.

That is, the top cover 1110 and the bottom layer 1140 may be affixed in order to form a chamber holding the components of the patch 1100 during shipping. To this end, the bottom layer 1140 may further include one or more adhesives (not shown) which are used to adhere the bottom layer 1140 to the top cover 1110. The top cover 1110 and the bottom layer 1140 may be separated in order to remove the patch 1100 from such a chamber such that remaining components (e.g., the electrode apparatus 1120 and the adhesive layer 1130) may be removed from the packaging when the electrode apparatus 1120 is to be applied to the skin of a user.

The adhesive layer 1130 covers a surface of the patch 1100 where the patch 1100 is to be attached to the skin of a user and may further be utilized to create an electrically insulating barrier between electrodes. Such an electrically insulating barrier may prevent liquids or other foreign materials from creating a short circuit. In some embodiments, the adhesive layer 1130 is made out of silicone, acrylic, hydrocolloid, polyeurethane, Tegaderm, and the like.

In some embodiments, an area of a portion of the adhesive surrounding each electrode of the electrode apparatus 1120 is around 0.5 centimeters thick. Such a 0.5 centimeter thick area around the portion of the adhesive may allow for holding the electrodes to the skin and creating a waterproof seal around the electrodes. In a further embodiment, a thickness of the adhesive is at least 5 millimeters in order to provide a waterproof barrier, a conduction barrier, or both.

In some embodiments, the adhesive layer 1130 may be further attached to a backing layer (not shown) that covers the adhesive until the patch 1100 is to be used. Such a backing layer may have a thickness of around 0.1 millimeters.

In some embodiments, the adhesive layer 1130 of the patch 1100 that interfaces with the skin may be made of two materials including an adhesive material and a conductive material (not shown), where the sections of the conductive material may act as electrodes to deliver electrical current in accordance with one or more disclosed embodiments. In such embodiments, the portion of the adhesive layer 1140 made of the adhesive material is between 0.1 and 1 millimeters thick.

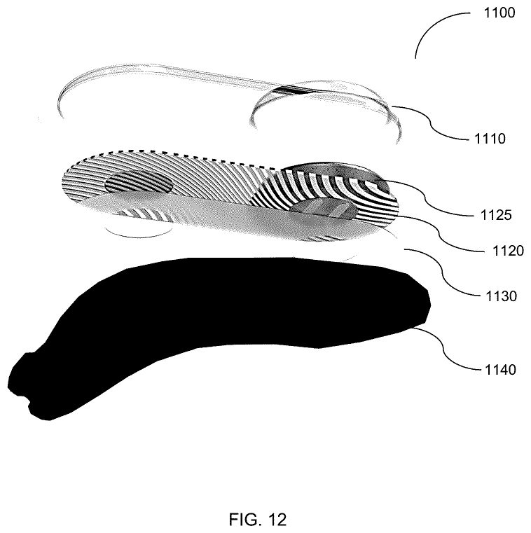

FIG. 12 is a bottom exploded view illustrating the components of the patch 1100 according to an embodiment.

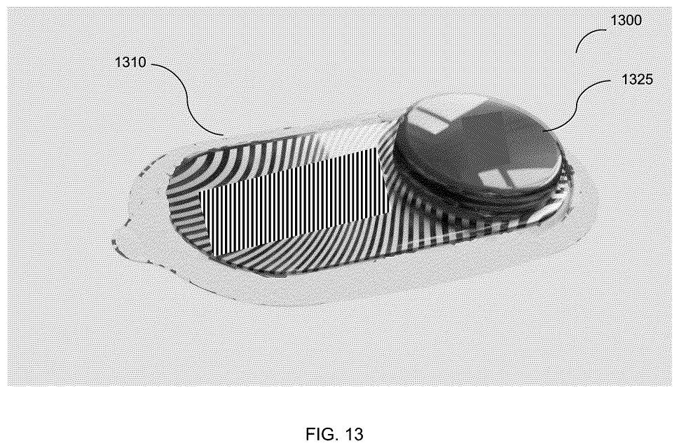

FIG. 13 is an assembled view 1300 of a device according to an embodiment. The assembled view 1300 illustrates a top cover 1310 and a housing 1325. The housing 1325 may include components as now described with respect to FIG. 14.

FIG. 14 is an exploded view of the housing 1325 according to an embodiment. The housing 1325 includes a connecting member 1410, a base 1420, a circuit board 1430, a battery 1440, and a cover 1450. The connecting member 1410 may be disposed on or attached to a device (e.g., the patch 700, FIG. 7), and may facilitate electrically connecting the circuit board 1430 with an electrode (e.g., the electrode 720 or the electrode 730, FIG. 7), for example via one or more wires as discussed above with respect to FIG. 6.

The base 1420 may be affixed to the cover 1450 in order to enclose the housing between the device and the connecting member 1410 with the cover 1450. The base 1420 and the cover 1450 may therefore be utilized to realize an enclosed cavity in which the other components of the device shown in FIG. 14 are disposed.

The circuit board 1430 may be configured to deliver electric pulses when the device is disposed on a nerve as described herein. To this end, the circuit board 1430 is electrically connected to the battery 1440. The battery 1440 provides electricity for distribution to electrodes (e.g., the electrodes 720 and 730 as shown in FIG. 7).

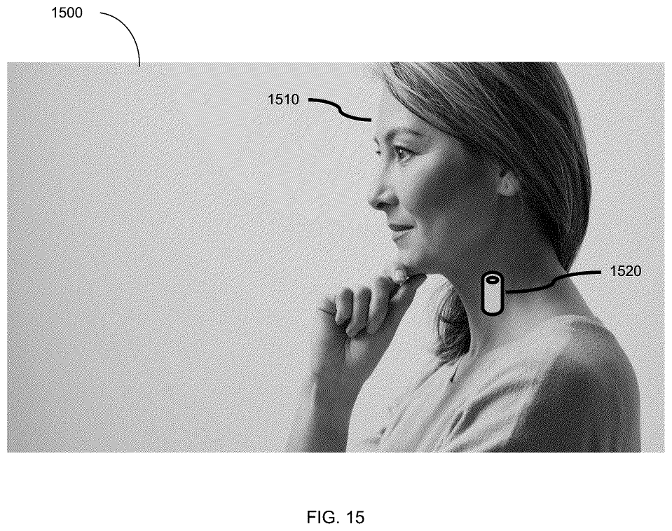

FIG. 15 is an illustration 1500 of a patch 1520 placed on a user 1510. The patch 1520 may be designed in accordance with one or more of the disclosed embodiments, for example, as described above with respect to FIGS. 11-14.

In some embodiments, a non-biphasic waveform may be utilized. As non-limiting examples, a monophasic waveform or a waveform which is otherwise of arbitrary shape may be utilized. In such embodiments, the general principles behind the device described herein may still be utilized for vagus nerve stimulation. More specifically, in a further embodiment, multiple temporally short (e.g., frequency above a predetermined threshold) waveforms may be delivered at the skin across multiple pairs of electrodes. Such an embodiment leverages principles of interferential current stimulation in order to allow for creating a wider waveform than would be created using a single pair of electrodes, where the wider waveform can be of any desired shape. In some embodiments, monophasic pulses are delivered in an alternating manner such that the total summation of charge delivered to the tissue is balanced (e.g., reduced as much as possible or otherwise reduced).

In this regard, it is noted that many peripheral nerves such as the vagus nerve are located close enough to the surface of the skin that those nerves can be stimulated transcutaneously. Transcutaneous stimulation may be performed by applying voltage differences across surface electrodes disposed on the skin. This creates an electrical field which penetrates through the skin and can deliver energy to neurons underneath the skin.

It is further noted that, when stimulating a nerve directly, the energy is usually delivered in discreate pulses interspaced with periods of quiescence. The change in the electrical field delivered to the target over time during each pulse is referred to as the stimulation waveform which may be measured in voltage, current, and the like. A biphasic waveform contains two phases without opposite polarity (e.g., with the same polarity for each phase), which may be utilized to realize activation by exposing the target to a varying electrical potential, while reducing the risk of charge buildup because the charge delivery during a first phase of the waveform is cancelled out during a second oppositely polarized phase of the waveform.

It has been shown that the width and frequency of stimulation waveforms influences their ability to penetrate through tissue and activate targets. For example, relatively wider biphasic pulses (e.g., total length of 1 ms) can often provide stronger or more reliable activation of target versus shorter pulses (e.g. total length of 100 us). However, in at least some implementations, it may not be possible or practical to deliver a wide biphasic waveform to surface electrodes on the skin. In this regard, it has been identified that shorter (i.e., higher frequency) waveforms penetrate tissue deeper, with less sensation on the skin and less unintended activation of surrounding muscle. In such implementations, it may be desirable to utilize shorter waveforms to carry electrical energy across the more superficial off target layers (e.g. skin, fascia, etc.) which may be utilized to provide more effective target activation with less nontarget activation and skin sensation. For example, 10 Kilohertz monophasic pulses with 100 microseconds pulse width create less cutaneous sensation than 1 Kilohertz or lower pulses with 1000 microsecond pulse widths.

Additionally, in some instances, it has been identified that splitting the individual phases of a biphasic pulse into small bursts of monophasic pulses can actually increase activation. To this end, in at least some embodiments, such smaller burst pulses may be utilized to deliver stimulation of the vagus nerve. In a further embodiment, inferential current stimulation may be utilized to deliver stimulation with a relatively wide pulse width at target that is created via the spatiotemporal summation of electrical fields induced by relatively short pulses delivered at the skin surface.

In this regard, it is noted that inferential current stimulation leverages the ability of electrical fields to sum at points where current paths of the currents overlap. The location at which such overlap occurs is referred to as the interference point. The resulting current and waveform caused by the sum of electric fields passing through the interference point is referred to as the interference current. For example, if two pairs of electrodes are spatially aligned so that the current path of one pair crosses through the current path of another pair, then the overlap of these current paths would create an interference point.

The spatial pathway of the current between a given pair of electrodes is defined by properties of the material that the current flows through, the placement of the electrodes, and the pattern of current sent through the electrodes. As a non-limiting example, when two electrodes are placed on the skin and current is run between the two electrodes, then the depth of penetration into the skin of the current at a point located between the two electrodes is increased when the amplitude of the current is increased or when the distance between the two electrodes is increased. Given this relationship between spatial pathways and these parameters, it has been identified that different spatial arrays may be defined using multiple electrode pairs which each deliver certain electrical stimulation waveforms so as to create an interference waveform at a certain location (e.g., a location along the vagus nerve).

In accordance with some embodiments, interferential current stimulation is utilized to allow for delivering continuous alternating current (AC) high frequency waveforms (e.g., greater than 5000 Hz) across multiple unique pairs of electrodes in a manner such that, at the point where each stimulating electrode pair's electrical fields interfere with the electrical fields of other electrode pairs in the tissue, the electrical fields sum spatiotemporally in order to create an interferential waveform that is a similar frequency stimulation but is amplitude modulated at a relatively lower frequency (e.g., less than 100 Hz) as the sperate electrical fields that make up the waveform shift in and out of phase. Modulating amplitude of the interference waveform improves ability to activate target over non-amplitude modulated high frequency waveforms and may allow for controlling the frequency of activation of the target. Such interferential current stimulation may allow for providing better transcutaneous current penetration due to greater tolerated intensities that penetrate deeper into the body given that high frequency stimulation at the skin has been identified to be more comfortable for subjects at the same amplitude intensity than lower frequency stimulations. As a non-limiting example, a frequency of a first waveform of 5000 Hertz and a frequency of a second waveform of 5100 Hertz would result in a carrier frequency of 5000 Hertz and a beat frequency equal to the difference (i.e., 100 Hertz). When such waveforms overlap, they interfere with each other in a manner that results in an interference pattern equal to a roughly continuous alternating current waveform at the carrier frequency whose amplitude is cyclically modulated at the beat frequency. This low-frequency-amplitude-modulated high frequency AC pattern may allow for improved activation of the target structure at the interference point.

In other embodiments, the interferential stimulation may be realized using intersectional short pulse transcutaneous direct current stimulation that uses discontinuous pulses delivered transcutaneously to produce, at an interference site, a continuous stimulation waveform. In a further embodiment, the electrical waveforms delivered by the electrode pairs used to create the interference pattern are pulsed waveforms which are discontinuous. This may allow for delivering continuous current stimulation at the interference point while allowing for duty-cycled discontinuous stimulation across electrode pairs, which in turn may aid in preventing heat or charge buildup at the surface electrode or in reducing sensation. As a non-limiting example, two pairs of electrodes each deliver a pulsed direct current (DC) signal that is temporally aligned such that each pair delivers a DC pulse subsequently after the other pair's latest DC pulse finishes. This results in a situation where, at either electrode pair, the DC current is only flowing half of the time while, at the interference point, there is continuous DC current due to the temporally aligned summation of the two pulsed currents that occurs at the interference point. Likewise, electrode pairs delivering separate pulses of AC current may be spatially and temporally aligned so as to create a continuous AC current at the interference site from a temporal summation of discontinuous AC currents delivered by the electrode pairs.

Accordingly, in some embodiments, multiple pairs of electrodes may be leveraged in order to provide interferential current stimulation. To this end, any or all of the devices as described herein may be realized using multiple electrode pairs. Multiple electrode pairs may be utilized to deliver relatively short width (i.e., high frequency) waveforms to create an interference pattern that results in a stimulation waveform at a target that is relatively wide (i.e., low frequency) as compared to individual stimulation waveforms delivered at the surface. This may be utilized to target the vagus nerve as described herein in order to stimulate the vagus nerve, thereby enhancing sensory processing. As noted above, such interferential current stimulation may be utilized to improve penetration of the skin while reducing sensation in order to optimize activation.

In accordance with various embodiments utilizing multiple electrode pairs, an interference pattern realized as a biphasic pulsed waveform with total pulse width between 200 and 2000 microseconds may be delivered in a discontinuous pulsed fashion (e.g., at 30 Hertz frequency). In a further embodiment, the interference point at which all electrical signals across each of the electrode pairs intersect is aligned with the location of the vagus nerve when applied to the surface of the skin at the carotid triangle in order to allow the currents having the resulting interference pattern to stimulate the vagus nerve. In yet a further embodiment, the interference-created biphasic waveform of the resulting interference pattern is delivered at a frequency of about 15 to 60 Hertz with quiescence in between pulses.

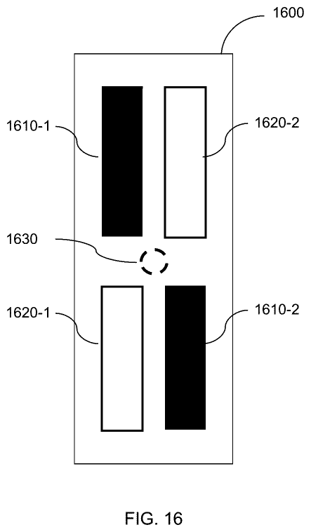

Embodiments in which multiple pairs of electrodes are utilized are now described with respect to FIGS. 16 through 19.

FIG. 16 is a schematic diagram illustrating a design of a device 1600 utilizing multiple pairs of electrodes 1610 and 1620 according to an embodiment. Each pair of electrodes 1610 and 1620 includes a respective first electrode 1610-1 or 1620-1 and a respective second electrode 1610-2 or 1620-2. Current paths between the respective first electrodes 1610-1 and 1620-1 with the second electrodes 1610-1 and 1620-1 cross at an interference point in the tissue of the skin of a subject (e.g., as marked by a circle marker 1630).

In an embodiment, a set of electrode pairs such as the two electrode pairs 1610 and 1620 depicted in FIG. 16 may be utilized to realize a device. In a further embodiment, such a device includes multiple electrode pairs which are spatially aligned such that the current paths of the electrode pairs intersect over a location of a stimulation target such as the vagus nerve. In yet a further embodiment, the amplitude of the current for each electrode pair is set independently in order to target a region that is off center of the interference point which would have been realized if current amplitude was equal across all electrode pairs. This follows as the amplitude of the current influences the depth of penetration and current path. Further, differences in frequency and waveform shape can be used to alter the location of the interference point.

In another embodiment, the waveforms delivered by each electrode pair are of a relatively short width such as, but not limited to, less than 100 microseconds, or between 100 and 200 microseconds (which results in a waveform frequency of over 10,000 or 5,000 Hertz, respectively). In some embodiments, the waveforms delivered by the electrode pairs are charge balanced. That is, in such embodiments, the charge delivered over the time length of each full waveform results in a charge of 0. This may be achieved by cancelling out a charge delivered via one polarity by delivering an opposite charge of equal amount using a current delivered with an opposite polarity. In some embodiments, the waveform may be asymmetrically balanced by up to 50% more charge delivered to one polarity. Such asymmetric balancing may be utilized, for example, to prevent voltage drift (e.g., change in the resting voltage potential of the tissue due to second phase interacting with tissue that has already had its voltage potential altered by the first phase). In some instances, the stimulation waveform may alternate between cathode leading and anode leading polarity to reduce risk of voltage drift.

In an embodiment, the waveforms delivered by the individual electrode pairs are shaped such that the temporally aligned summation at the interference point results in a relatively wide waveform delivered to the stimulation target at that interference point. That is, the resulting waveform of the interference pattern is temporally wider than any of the discrete waveforms delivered across the individual electrode pairs. Such a wider width waveform may have a width between 200 to 1 microsecond (us) per phase and could be the result of interference of waveforms delivered across electrode pairs that were relatively shorter width (i.e., less than 100 us per phase).

In some embodiments, the waveforms delivered by the individual electrode pairs are shaped so that the resulting temporally summed waveform at the interference point is charge balanced. In a further embodiment, such a charge balanced waveform is a biphasic waveform. In yet a further embodiment, the temporally summed waveform at the interference point is asymmetrically balanced by up to 50% more charge delivered to one polarity, which may allow for preventing voltage drift.

In some embodiments, the electrodes at a cranial edge of the patch are cathode leading. Such use of cathode leading electrodes at the cranial edge of the patch may allow for directing stimulation in an optimal direction (i.e. efferent signals to brain). In other embodiments, if an efferent signal away from the brain is desired, electrodes at the cranial edge of the patch are anode leading.

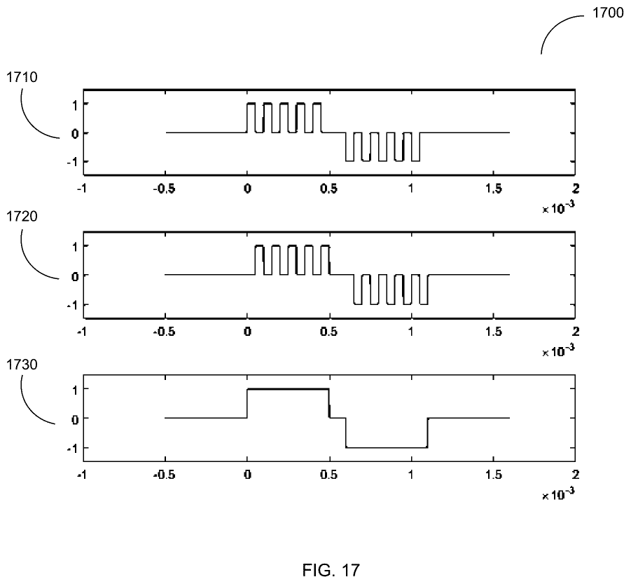

FIG. 17 is a waveform diagram 1700 illustrating current waveforms delivered across two electrode pairs which may be utilized in accordance with certain embodiments. The waveform diagram 1700 depicts waveforms which may be realized, for example, using the electrode pairs depicted in FIG. 16.

The waveform diagram 1700 illustrates graphs 1710, 1720, and 1730, of three waveforms. More specifically, the graphs 1710 and 1720 depict waveforms of respective electrode pairs, the graph 1730 depicts a waveform of the summed interference pattern which occurs at the interference site between the electrode pairs. As depicted in FIG. 17, short monophasic pulses by the electrode pairs may be utilized to generate a wider biphasic pulse at the interference point.

The X-axis for each of the graphs 1710 through 1730 represents time in seconds, and the Y-axis for each graph represents a strength of an electric field as measured in current amplitude or voltage. In the example depicted in FIG. 16, when a biphasic stimulation pulse is to be delivered to the vagus nerve, a first pair of electrodes deliver a current pattern as depicted in the graph 1710 and a second pair of electrodes deliver a current pattern as depicted in the graph 1720.

The current pattern depicted in the example graph 1710 includes a series of monophasic pulses occurring over 1 millisecond in time, each pulse being about 0.1 millisecond wide, with a 0.1 millisecond gap of quiescence between pulses and a polarity of pulses switching after the first 0.5 milliseconds. In some embodiments, a 0.1 millisecond gap may be inserted between the first and second phase of the waveform in order to create an interphase gap which improves activation as discussed above.

In the example depicted in FIG. 17 the second electrode pair whose waveform is depicted in the graph 1720 delivers a current pattern which is essentially the same pattern delivered by the first electrode pair, time shifted by 0.1 millisecond. This time shift temporally aligns the patterns such that current only flows through one electrode pair at a time. In other embodiments (not shown in FIG. 17), the current between the electrodes may overlap at least partially.

The resulting biphasic waveform depicted in the graph 1730 of FIG. 17 has phases which are 500 microseconds in width and a 100-microsecond interphase gap of quiescence. Such an interphase gap may aid in activating a neural target. In other embodiments (not shown in FIG. 17), such an interphase gap may be excluded.

It should be noted that the example shown in FIG. 17 depicts an interphasic gap in stimulation between phases, but that such an interphasic gap may be avoided in at least some embodiments. In some embodiments, different high frequency waveforms having relatively short width phases (e.g., less than 100 microseconds per phase or frequency less than 5 Kilohertz) may be utilized in order to produce a relatively wide biphasic pulse (e.g., greater than 200 microseconds per phase). This allows for delivering a wide biphasic waveform to the target for activation while reducing exposure of the surface skin and surrounding muscle to relatively shorter width pulses that are less likely to cause sensation of the skin or activation of non-target muscles.

It should be noted that two electrode pairs are illustrated and described with respect to FIGS. 16 and 17, but additional pairs of electrodes (e.g., 3, 4, or more) may be utilized in accordance with at least some disclosed embodiments. In such embodiments utilizing more than two electrode pairs, the pulse width delivered by each electrode pair may be shortened and the off period between monophasic pulses may be lengthened. As a non-limiting example, a device with 4 pairs of electrodes may deliver 0.05 millisecond pulses with 0.15 milliseconds of quiescence between pulses. When each of the 4 pairs of electrodes emit pulses sequentially, then the patterns would temporally align such that only one electrode pair is delivering current at any given time while realizing the wider biphasic waveform of 200 microseconds or greater per phase at the interference site.

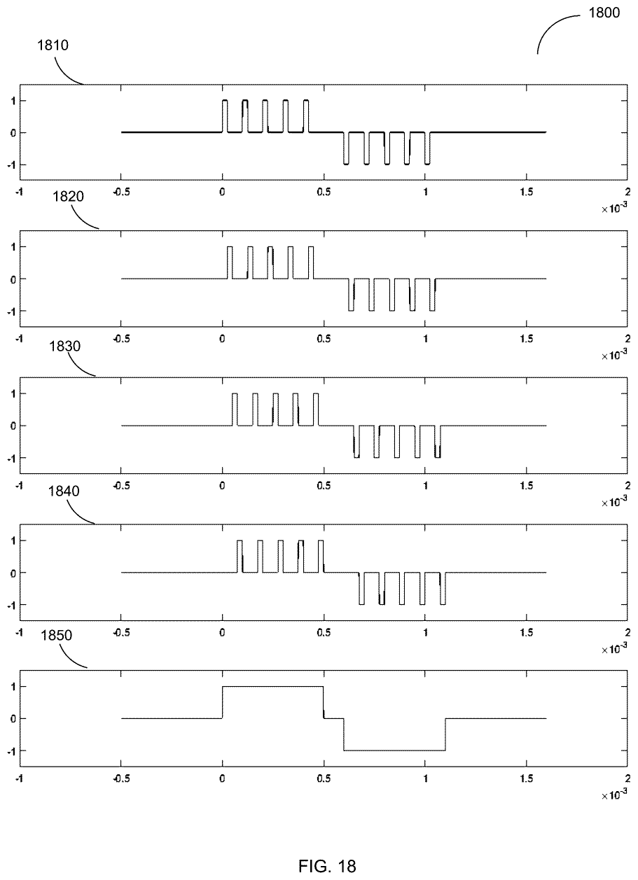

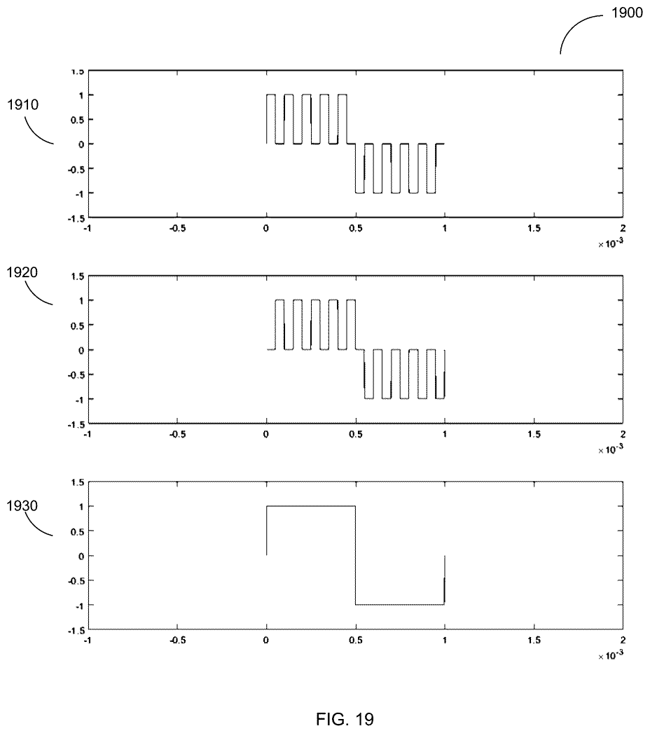

An example illustrating a four electrode pair configuration is demonstrated by FIG. 18. FIG. 18 is a waveform diagram 1800 illustrating current waveforms delivered across four electrode pairs which may be utilized in accordance with certain embodiments. The waveform diagram 1800 includes graphs 1810 through 1850. Each of the graphs 1810 through 1840 illustrates a waveform of a respective electrode pair, and the graph 1850 illustrates the resulting summed biphasic waveform at the interference point.

In some embodiments, the current patterns to be delivered by each electrode pair are computationally generated. This may be achieved, for example, by deconvolving a biphasic waveform to be generated at the interference point into multiple individual waveforms that, when temporally summed at the interference point, produces the biphasic waveform to be generated. In these instances, the individual electrical fields would be designed to be higher frequency than the resulting summed interference waveform (e.g., exhibiting the majority of its frequency power spectrum above 5000 Hz).

In some embodiments, the biphasic waveform created at the interference point resembles a sinusoid shape instead of square pulses. In such an embodiment, the phase width equals half of the length of a full cycle.

In some embodiments, in addition to the electrode pairs used to create the interferential biphasic stimulation, one or more additional pairs of electrodes may be utilized to deliver a steady direct current stimulation at a low amplitude (e.g., an amplitude between 0.1 and 3 milliamps) in order to induce an ephaptic effect at the target location (i.e., the interference point). This may allow for increasing the likelihood of neural tissue being activated by the co-occurring biphasic stimulation pulses.

FIG. 19 is a waveform diagram 1900 illustrating a summed biphasic waveform at an interference point. Specifically, FIG. 19 depicts graphs 1910 through 1930, where the graphs 1910 and 1920 illustrate waveforms of individual electrode pairs and the graph 1930 illustrates a resulting summed waveform at the interference point. As depicted in FIG. 19, the graphs 1910 and 1920 do not demonstrate an interphase gap, as contrasted with the waveforms depicted in FIG. 17.

In some embodiments utilizing multiple electrode pairs in which target locations to be stimulated are within the neck, multiple electrode contact points that wrap around the neck may be utilized. Such wrapping contact points may allow for the creation of three-dimensional electric fields that leverage the properties discussed above regarding interferential stimulation in order to stimulate target nerves, muscles, and the like. It is noted that, because the interference point between electrode pairs is determined by the spatial location of the electrodes and the relative intensities of the currents being delivered through the electrodes, the number of unique current pathways may be increased by utilizing electrodes disposed at multiple locations wrapping around the neck.

In such an implementation where electrodes are to be wrapped around the neck, the device may be realized as a collar or cuff worn around the neck which wraps at least partially around the neck. To this end, in an embodiment, electrodes with a diameter between 1 and 3 centimeters may be utilized with interelectrode distances between 3 and 5 centimeters. A height of such a collar may be around 2 to 5 inches in order to fit a human neck, with a circumference of around 10 to 20 inches. Such a device may include a power source, controller, and electrodes as discussed above. In this embodiment, the controller may control the current being delivered across each individual electrode pair separately as discussed above with respect to interferential current stimulation in order to deliver discrete biphasic stimulation waveforms to target locations (e.g., interference points). In some embodiments, an adhesive layer on the device may be used to adhere the device to the neck. In other embodiments, a diameter of the device may be cinched so as to provide a friction fit when the device is placed on the neck.

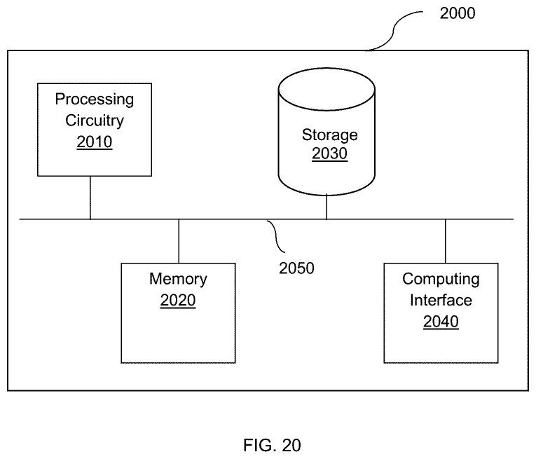

FIG. 20 is a schematic diagram of a hardware layer 2000 of computing components of a device according to an embodiment. The hardware layer 2000 includes a processing circuitry 2010 coupled to a memory 2020, a storage 2030, and a computing interface 2040. In an embodiment, the components of the hardware layer 2000 may be communicatively connected via a bus 2050.

The processing circuitry 2010 may be realized as one or more hardware logic components and circuits. For example, and without limitation, illustrative types of hardware logic components that can be used include field programmable gate arrays (FPGAs), application-specific integrated circuits (ASICs), Application-specific standard products (ASSPs), system-on-a-chip systems (SOCs), graphics processing units (GPUs), tensor processing units (TPUs), general-purpose microprocessors, microcontrollers, digital signal processors (DSPs), and the like, or any other hardware logic components that can perform calculations or other manipulations of information.

The memory 2020 may be volatile (e.g., random access memory, etc.), non-volatile (e.g., read only memory, flash memory, etc.), or a combination thereof.

In one configuration, software for implementing one or more embodiments disclosed herein may be stored in the storage 2030. In another configuration, the memory 2020 is configured to store such software. Software shall be construed broadly to mean any type of instructions, whether referred to as software, firmware, middleware, microcode, hardware description language, or otherwise. Instructions may include code (e.g., in source code format, binary code format, executable code format, or any other suitable format of code). The instructions, when executed by the processing circuitry 2010, cause the processing circuitry 2010 to perform the various processes described herein.

The storage 2030 may be magnetic storage, optical storage, and the like, and may be realized, for example, as flash memory or other memory technology, compact disk-read only memory (CD-ROM), Digital Versatile Disks (DVDs), or any other medium which can be used to store the desired information.

The computing interface 2040 allows the hardware layer 2000 to communicate with other systems, devices, components, applications, or other hardware or software components, for example as described herein. In some implementations, the computing interface 2040 may be or may include a network interface.

It should be understood that the embodiments described herein are not limited to the specific architecture illustrated in FIG. 20, and other architectures may be equally used without departing from the scope of the disclosed embodiments.

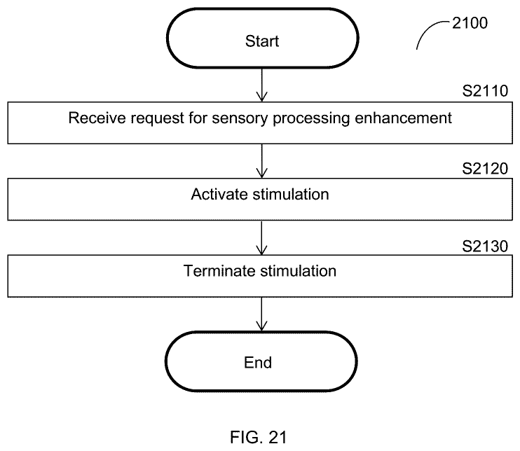

FIG. 21 is a flowchart 2100 illustrating a method for vagus nerve stimulation according to an embodiment. In an embodiment, the method is performed via the hardware layer 2000, FIG. 20. In a further embodiment, the hardware layer is included in a device as described in one or more of the disclosed embodiments.

At S2110, a request for sensory processing enhancement is received. The request may be received, for example, based on user inputs (e.g., inputs indicating a user request for sensory processing enhancement), based on an automated program (e.g., a program configured to detect hearing loss), and the like. In some embodiments, the request may further indicate a degree of stimulation to be applied. User inputs may be received, for example but not limited to, from a user device of the user (e.g., via a software application installed on the user device), or via one or more buttons or other physical components of the device. The request for sensory processing may further indicate timing, duration, or both, for the stimulation.

At S2120, stimulation is activated. In an embodiment, S2120 includes sending one or more signals including commands for stimulation to one or more components of the device configured to provide nerve stimulation. In particular, such components may be or may include one or more components (e.g., electrodes) configured to apply electrical currents to the user in order to stimulate sensory processing as discussed herein.

In an embodiment, the device is a device configured in accordance with one or more disclosed embodiments. In a further embodiment, the device is disposed on one or more arteries of the user. In yet a further embodiment, the device is disposed on a carotid triangle of a neck of the user. In any such embodiments, the device is configured for vagus nerve stimulation as described herein.

In an embodiment, the stimulation is delivered using an uninterrupted tonic stimulation pattern. As noted above, such a pattern may improve effects on sensory perception, for example as compared to a duty-cycled stimulation pattern or other alternating or fluctuating stimulation pattern. Such stimulation may be delivered to the vagus nerve in order to steadily activate the norepinephrine (NE) system in a manner that improves the ability of a brain of the user to accurately process sensory information.

In an embodiment, stimulation is applied using a continuous stimulation pattern for a time period between 1 and 12 hours. In a further embodiment, a maximum length of time of quiescence is 10 seconds during the time period in which the continuous stimulation pattern is applied. That is, the maximum duration of any period of inactive time (i.e., time in which pulses are not being emitted) during the stimulation is 10 seconds. In another embodiment, such a maximum length of time of quiescence is 100 milliseconds during the time period in which the continuous stimulation pattern is applied.

At S2130, a trigger for ceasing sensory processing enhancement is detected. Such a trigger may be or may include, but is not limited to, passage of a predetermined period of time since beginning enhancement, passage of a duration of time indicated in the request, an end of detection of sensory signals, a combination thereof, and the like.

It is important to note that the embodiments disclosed herein are only examples of the many advantageous uses of the innovative teachings herein. In general, statements made in the specification of the present application do not necessarily limit any of the various claimed embodiments. Moreover, some statements may apply to some inventive features but not to others. In general, unless otherwise indicated, singular elements may be in plural and vice versa with no loss of generality. In the drawings, like numerals refer to like parts through several views.

At least some embodiments disclosed herein can be implemented as hardware, firmware, software, or any combination thereof. Moreover, the software may be implemented as an application program tangibly embodied on a program storage unit or computer readable medium consisting of parts, or of certain devices and/or a combination of devices. The application program may be uploaded to, and executed by, a machine comprising any suitable architecture. Preferably, the machine is implemented on a computer platform having hardware such as one or more central processing units (“CPUs”), a memory, and input/output interfaces. The computer platform may also include an operating system and microinstruction code. The various processes and functions described herein may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium is any computer readable medium except for a transitory propagating signal.

All examples and conditional language recited herein are intended for pedagogical purposes to aid the reader in understanding the principles of the disclosed embodiment and the concepts contributed by the inventor to furthering the art, and are to be construed as being without limitation to such specifically recited examples and conditions. Moreover, all statements herein reciting principles, aspects, and embodiments of the disclosed embodiments, as well as specific examples thereof, are intended to encompass both structural and functional equivalents thereof. Additionally, it is intended that such equivalents include both currently known equivalents as well as equivalents developed in the future, i.e., any elements developed that perform the same function, regardless of structure.

It should be understood that any reference to an element herein using a designation such as “first,” “second,” and so forth does not generally limit the quantity or order of those elements. Rather, these designations are generally used herein as a convenient method of distinguishing between two or more elements or instances of an element. Thus, a reference to first and second elements does not mean that only two elements may be employed there or that the first element must precede the second element in some manner. Also, unless stated otherwise, a set of elements comprises one or more elements.

As used herein, the phrase “at least one of” followed by a listing of items means that any of the listed items can be utilized individually, or any combination of two or more of the listed items can be utilized. For example, if a system is described as including “at least one of A, B, and C,” the system can include A alone; B alone; C alone; 2A; 2B; 2C; 3A; A and B in combination; B and C in combination; A and C in combination; A, B, and C in combination; 2A and C in combination; A, 3B, and 2C in combination; and the like.

What is claimed is:

1. A device, comprising:
a power source;
a plurality of pairs of electrodes having a plurality of current paths, wherein each pair of electrodes is electrically connected to the power source, wherein each pair of electrodes is adapted to apply a current delivered from the power source in a respective current path of the plurality of current paths in order to stimulate a subcutaneous target within a subject, wherein the plurality of current paths have a plurality of first waveforms with a plurality of first frequencies, wherein the plurality of current paths overlap at an interference point having a second waveform with a second frequency, wherein the second waveform is result of a summation of electrical fields of the current delivered via each pair of electrodes; and
a controller, wherein the controller is communicatively connected to the power source, wherein the controller is configured to send control signals in order to control the current delivered from the power source to each pair of electrodes in order to create a stimulation pattern for each electrode pair, wherein the controller is further configured to control the current delivered to each pair of electrodes such that each of the plurality of first frequencies is higher than the second frequency and such that the second waveform includes discrete interference pulses delivered with periods of quiescence between the interference pulses.

2. The device of claim 1, wherein the current delivered via each pair of electrodes is realized via electrical signals that are between 200 and 2000 microseconds in length.

3. The device of claim 1, wherein the second waveform is charge balanced.

4. The device of claim 1, wherein an interference pattern generated via the overlap of the plurality of current paths of the plurality of pairs of electrodes is a biphasic waveform.

5. The device of claim 4, wherein the biphasic waveform includes an interphase gap.

6. The device of claim 1, wherein the second waveform has a total pulse width of 200 to 2000 microseconds.

7. The device of claim 6, wherein the total pulse width of the second waveform is 1 millisecond.

8. The device of claim 1, wherein the discrete stimulation pulses of the interference pattern is delivered at a frequency between 15 and 60 Hertz.

9. The device of claim 1, wherein the discrete stimulation pulses of the interference pattern is delivered at a frequency below 100 Hertz.

10. The device of claim 1, wherein each of the plurality of first frequencies is above a predetermined threshold.

11. The device of claim 10, wherein the predetermined threshold is 5,000 Hertz.

12. The device of claim 1, wherein each of the plurality of first frequencies is between 5,000 Hertz and 10,000 Hertz.

13. The device of claim 1, wherein each of the plurality of first frequencies is above 10,000 Hertz.

14. The device of claim 1, wherein a width of a pulse of each of the plurality of first frequencies is 200 microseconds.

15. The device of claim 1, wherein a width of a pulse of each of the plurality of first frequencies is between 50 and 200 microseconds.

16. The device of claim 1, wherein a width of a pulse of each of the plurality of first frequencies is less than 100 microseconds.

17. The device of claim 1, wherein a charge delivered over a time length of each of the plurality of first waveforms is zero.

18. The device of claim 1, wherein a charge delivered via each of the plurality of first waveforms is asymmetrically balanced.

19. The device of claim 1, wherein a charge delivered over a time length of the second waveform is zero.

20. The device of claim 1, wherein a charge delivered via the second waveform is asymmetrically balanced.

21. The device of claim 1, wherein each of the plurality of first waveforms is a discontinuous waveform, wherein the second waveform is a continuous waveform.

22. The device of claim 1, wherein each of the plurality of pairs of electrodes has an interelectrode distance, wherein the interelectrode distance of each of the plurality of pairs of electrodes is defined based on a predetermined carotid triangle height.

23. The device of claim 22, wherein the interelectrode distance of each of the plurality of pairs of electrodes is between 3 and 7 centimeters.

24. The device of claim 23, wherein each electrode among the plurality of electrode pairs has a diameter between 1 and 3 centimeters.

25. The device of claim 1, further comprising:
a resistive sensor adapted to measure resistance of the device; and
a switch, wherein the switch is configured to turn on when the resistance measured by the resistive sensor is above a predetermined threshold.

26. The device of claim 1, further comprising:
an impedance sensor adapted to measure impedance of the device; and
a switch, wherein the switch is configured to turn on when the impedance measured by the impedance sensor is within a predetermined range.

27. The device of claim 1, wherein the controller is further configured to cause the current delivered from the power source to each of the pair of electrodes at a first current level and then to incrementally increase the current from the first current level to a second current level.

28. The device of claim 1, further comprising:
an adhesive layer, wherein the adhesive layer is adapted to adhere to a surface of skin.

29. The device of claim 1, wherein each pair of electrodes is adapted to apply a current delivered from the power source in order to stimulate a vagus nerve of the subject.

30. A non-transitory computer readable medium having stored thereon instructions for causing a processing circuitry to execute a process, the process comprising:
sending control signals in order to control current delivered from a power source to a plurality of pairs of electrodes, wherein each pair of electrodes is adapted to apply a current delivered from the power source in a respective current path of the plurality of current paths in order to stimulate a vagus nerve of a subject, wherein the plurality of current paths have a plurality of first waveforms with a plurality of first frequencies, wherein the plurality of current paths overlap at an interference point having a second waveform with a second frequency, wherein the control signals are sent such that each of the plurality of first frequencies is higher than the second frequency.

31. A system for interferential current stimulation, comprising:
a processing circuitry; and
a memory, the memory containing instructions that, when executed by the processing circuitry, configure the system to:
send control signals in order to control current delivered from a power source to a plurality of pairs of electrodes, wherein each pair of electrodes is adapted to apply a current delivered from the power source in a respective current path of the plurality of current paths in order to stimulate a vagus nerve of a subject, wherein the plurality of current paths have a plurality of first waveforms with a plurality of first frequencies, wherein the plurality of current paths overlap at an interference point having a second waveform with a second frequency, wherein the control signals are sent such that each of the plurality of first frequencies is higher than the second frequency.
